(19) United States
(12) Patent Application Publication
(10) Pub. No.: US 2014/0046218 A1
(43) Pub. Date: Feb. 13, 2014

(54) VIBRATION DETECTING APPARATUS, VIBRATION DETECTING METHOD, VIBRATION DETECTING SYSTEM, AND PROGRAM

(71) Applicant: SONY CORPORATION, Tokyo (JP)

(72) Inventors: Juri SAKAI, Tokyo (JP); Noriaki FUJITA, Tokyo (JP)

(21) Appl. No.: 13/908,180

(22) Filed: Jun. 3, 2013

(30) Foreign Application Priority Data
Aug. 10, 2012 (JP) 2012178558

(51) Int. Cl. A61B 7/04

(52) U.S. Cl. CPC A61B 7/04
USPC 600/586

(57) ABSTRACT

There is provided a vibration detecting apparatus including a biological vibration detecting unit that is capable of detecting a biological vibration, and a correction filter that corrects a frequency characteristic and a phase characteristic of a vibration signal obtained by the biological vibration detecting unit with at least an inverse characteristic of a characteristic of the biological vibration detecting unit.

### BACKGROUND

The present disclosure relates to a vibration detecting apparatus, a vibration detecting method, a vibration detecting system, and a program and more particularly, to a vibration detecting apparatus that detects a biological vibration including a cardiac sound and a pulmonary sound.

In order to improve auscultatory ability using stethoscopes, it is necessary to hear an auscultatory sound repetitively by a medical examination and training. However, because the stethoscopes are different in acoustic characteristics due to a performance difference between products and a structural problem, it is difficult to diagnose patients using stethoscopes other than familiar stethoscopes.

FIG. 53 illustrates a configuration example of an analog stethoscope 500 according to the related art. The analog stethoscope 500 mainly includes a chest piece 501, a rubber tube 502, ear tubes 503, and ear pieces 504 and has a characteristic deteriorated at each portion through which a sound propagates.

A diaphragm on the chest piece 501 or the ear piece 504 individually has a frequency characteristic and the rubber tube 502 or the ear tube 504 causes resonance. A shape or a material of the diaphragm is different for each maker or model, which results in causing an individual difference. In addition, a length and a bore of the rubber tube 502 or the ear tube 503 are different and a resonance point changes, which may result in affecting a medical examination.

Recently, digital stethoscopes that have functions of amplifying sound, reducing noise, and improving clarity have been suggested. The digital stethoscopes according to the related art mainly have a structure similar to the structure of the analog stethoscopes (for example, Japanese Patent Application Laid-Open (JP-A) No. 2007-275324).

### SUMMARY

As described above, because the digital stethoscopes according to the related art mainly have the structure similar to the structure of the analog stethoscopes, there is a problem in that the characteristic is deteriorated at each portion through which the sound propagates, similar to the analog stethoscopes.

It is desirable to enable a biological vibration including a cardiac sound and a pulmonary sound to be detected with a superior characteristic.

According to an embodiment of the present technology, there is provided a vibration detecting apparatus including a biological vibration detecting unit that is capable of detecting a biological vibration, and a correction filter that corrects a frequency characteristic and a phase characteristic of a vibration signal obtained by the biological vibration detecting unit with at least an inverse characteristic of a characteristic of the biological vibration detecting unit.

In the present disclosure, the vibration detecting apparatus includes the biological vibration detecting unit. The biological vibration detecting unit is configured to be capable of detecting the biological vibration. The biological vibration includes an organ sound such as a cardiac sound and a pulmonary sound, a respiratory sound such as a snoring sound, and other biological vibrations. The biological vibration detecting unit has a configuration in which a microphone is mounted on a chest piece. In addition, the biological vibration detecting unit may include an acceleration sensor that is used in a state in which the acceleration sensor directly adheres closely to a skin and a sensor that detects a vibration from a reflection wave such as a laser or a supersonic wave.

By the correction filter, the frequency characteristic and the phase characteristic of the vibration signal obtained by the biological vibration detecting unit are corrected with at least the inverse characteristic of the characteristic of the biological vibration detecting unit. For example, the correction filter may be a filter that has a constant group delay characteristic. As the filter having the constant group delay characteristic, a finite impulse response (FIR) filter is exemplified. In this case, the acoustic characteristic can be corrected without generating phase characteristic distortion.

For example, the correction filter may be a multi-stage filter that includes a predetermined number of static filters having a fixed filter characteristic and a predetermined number of dynamic filters having a variable filter characteristic. The filter characteristic of the dynamic filter is changed by a manual operation of a user or is automatically changed according to information of environment and shape changes.

As such, in the present disclosure, the frequency characteristic and the phase characteristic of the vibration signal that is obtained by the detection of the biological vibration detecting unit are corrected with at least the inverse characteristic of the characteristic of the biological vibration detecting unit. For this reason, the biological vibration can be detected with a superior characteristic without being affected by the acoustic characteristic (the frequency characteristic and the phase characteristic) of the biological vibration detecting unit.

In the present disclosure, the vibration detecting apparatus may further include a filter characteristic switching unit that switches a filter characteristic of the correction filter. For example, the filter characteristic switching unit may switch the filter characteristic using a filter coefficient downloaded from an external apparatus connected to a network. For example, the filter characteristic switching unit may switch the filter characteristic using a filter coefficient extracted from a filter coefficient storage unit. In this case, the biological vibration can be detected with a superior characteristic, according to a kind of the biological vibration or the living body.

In the present disclosure, the vibration detecting apparatus may further include a sound output unit that outputs a sound corresponding to the vibration signal corrected by the correction filter. A user can hear a biological vibration sound having a superior characteristic. In this case, the biological vibration detecting unit may have a plurality of independent detecting units, the correction filter may correct vibration signals obtained by the plurality of detecting units with respective filter characteristics, and the sound output unit may selectively output at least sounds corresponding to the plurality of vibration signals corrected by the correction filter.

In the present disclosure, the vibration detecting apparatus may further include a display unit that displays a waveform and/or a frequency spectrum corresponding to the vibration signal corrected by the correction filter. The user can observe a waveform or a frequency spectrum of a biological vibration sound such as a cardiac sound or a pulmonary sound with a superior characteristic.

In the present disclosure, the vibration detecting apparatus may further include a wireless transmitting unit that wirelessly transmits the vibration signal corrected by the correction filter to a predetermined number of external apparatuses. In this case, a biological vibration signal having a superior characteristic can be transmitted to external apparatuses. For example, the external apparatuses include a sound output apparatus such as a headphone, a display apparatus that displays a waveform or a frequency spectrum and an electronic medical chart generating apparatus and a measurement supporting apparatus that use the vibration signal.

For example, when a first, external apparatus and a second external apparatus are included as the external apparatuses, the wireless transmitting unit may selectively perform wireless transmission with respect, to the second external apparatus, based on an operation signal in the first external apparatus. In this case, a person (for example, a doctor and a teacher) who wears a headphone to be the first external apparatus and hears a biological vibration sound can arbitrarily set whether or not to allow a person (for example, a patient and a student) who wears a headphone to be the second external apparatus to hear the biological vibration sound.

Further, according to another embodiment of the present technology, there is provided a vibration detecting apparatus including a wireless receiving unit that receives a vibration signal obtained by detection of a biological vibration detecting unit, and a correction filter that corrects a frequency characteristic and a phase characteristic of the received vibration signal with at least an inverse characteristic of a characteristic of the biological vibration detecting unit.

In the present disclosure, the vibration signal obtained by the detection of the biological vibration detecting unit is received by the wireless receiving unit. In this case, the biological vibration includes an organ sound such as a cardiac sound and a pulmonary sound, a respiratory sound such as a snoring sound, and other biological vibrations. By the correction filter, the frequency characteristic and the phase characteristic of the vibration signal are corrected with at least the inverse characteristic of the characteristic of the biological vibration detecting unit. For example, the correction filter may be a filter that has a constant group delay characteristic. As the filter having the constant group delay characteristic, the FIR filter is exemplified.

As such, in the present disclosure, the frequency characteristic and the phase characteristic of the received vibration signal are corrected with at least the inverse characteristic of the characteristic of the biological vibration detecting unit. For this reason, the biological vibration signal can be obtained with a superior characteristic without being affected by the acoustic characteristic (the frequency characteristic and the phase characteristic) of the biological vibration detecting unit.

In the present disclosure, the vibration detecting apparatus may further include a filter characteristic switching unit that switches a filter characteristic of the correction filter. For example, the filter characteristic switching unit may switch the filter characteristic using a filter coefficient downloaded from an external apparatus connected to a network. For example, the filter characteristic switching unit may switch the filter characteristic using a filter coefficient extracted from a filter coefficient storage unit. In this case, the biological vibration signal can be obtained with a superior characteristic, according to a kind of the biological vibration or the living body.

For example, when the wireless receiving unit is wirelessly connected to a wireless transmitting apparatus transmitting the vibration signal, the filter characteristic switching unit may acquire filter characteristic information of the correction filter from the wireless transmitting apparatus and switch the filter characteristic of the correction filter.

Further, according to another embodiment of the present technology, there is provided a vibration detecting apparatus including a vibration signal acquiring unit that acquires a vibration signal obtained by detection of a biological vibration detecting unit, and a signal processing unit that outputs a result that is obtained by performing filtering of a correction filter correcting a frequency characteristic and a phase characteristic with at least an inverse characteristic of a characteristic of the biological vibration detecting unit with respect to the vibration signal. The signal processing unit includes a communication unit that performs communication for the filtering with an external apparatus connected to a network.

In the present disclosure, the vibration signal obtained by the detection of the biological vibration detecting unit is acquired by the vibration signal acquiring unit. For example, the vibration signal acquiring unit includes the biological vibration detecting unit or a wireless receiving unit wirelessly receiving a vibration signal obtained by the biological vibration detecting unit.

The result that is obtained by performing the filtering of the correction filter correcting the frequency characteristic and the phase characteristic with at least the inverse characteristic of the characteristic of the biological vibration detecting unit with respect to the vibration signal is output the signal processing unit. In this case, the signal processing unit has the communication unit that performs communication for the filtering with the external apparatus connected to the network. For example, the communication unit transmits the acquired vibration signal to the external apparatus and receives the result obtained by performing the filtering, from the external apparatus.

As such, in the present disclosure, in the signal processing unit, the communication for the filtering with the external apparatus connected to the network is performed and the result that is obtained by performing the filtering of the correction filter characteristic including the inverse characteristic of the characteristic of the biological vibration detecting unit with respect to the acquired vibration signal is obtained. For this reason, a biological vibration signal having a superior characteristic can be obtained without providing a correction filter having a heavy processing load in the signal processing unit.

According to the embodiments of the present disclosure described above, a biological vibration including a cardiac sound and a pulmonary sound can be detected with a superior characteristic.

### BRIEF DESCRIPTION OF THE DRAWINGS

FIG. 1 is a diagram illustrating a configuration example of a vibration detecting apparatus according to a first embodiment;

FIG. 2 is a block diagram illustrating a configuration example of an acoustic processing unit constituting a vibration detecting apparatus;

FIG. 3 is a diagram illustrating an example of an acoustic characteristic Hm of a chest piece;

FIG. 4 is a diagram illustrating an example of an inverse characteristic Hm⁻¹ of an acoustic characteristic Hm of a chest piece;

FIG. 5 is a diagram illustrating a relation of an impulse signal, an impulse response collected by a microphone having an output characteristic Hm, and an impulse signal obtained by filtering the impulse response by a filter having an inverse characteristic Hm⁻¹;

FIG. 6 is a diagram illustrating an example of the case in which a filter processing unit has a multi-stage filter as a correction filter;

FIG. 7 is a flowchart illustrating a processing sequence of an acoustic processing unit;

FIG. 8 is a diagram illustrating an example of a processing sequence in the case in which convolution processing is executed on a frequency axis;

FIG. 9 is a diagram illustrating a configuration example of a vibration detecting apparatus according to a second embodiment;

FIG. 10 is a block diagram illustrating an acoustic processing unit and a headphone constituting a vibration detecting apparatus;

FIG. 11 is a diagram illustrating a configuration example of a vibration detecting apparatus according to a third embodiment;

FIG. 12 is a block diagram illustrating a configuration example of an acoustic processing unit and a display device constituting a vibration detecting apparatus;

FIG. 13 is a flowchart illustrating a processing sequence of an acoustic processing unit and a display device;

FIG. 14 is a block diagram illustrating another configuration example of an acoustic processing unit and a display device constituting a vibration detecting apparatus;

FIG. 15 is a flowchart illustrating a processing sequence of an acoustic processing unit and a display device;

FIG. 16 is a diagram illustrating a configuration example of a vibration detecting apparatus according to a fourth embodiment;

FIG. 17 is a block diagram illustrating a configuration example of an acoustic processing unit and a headphone constituting a vibration detecting apparatus;

FIG. 18 is a flowchart illustrating a processing sequence of an acoustic processing unit;

FIG. 19 is a flowchart illustrating a processing sequence of a headphone;

FIG. 20 is a block diagram illustrating another configuration example of an acoustic processing unit and a headphone constituting a vibration detecting apparatus;

FIG. 21 is a flowchart illustrating a processing sequence of an acoustic processing unit;

FIG. 22 is a flowchart illustrating a processing sequence of a headphone;

FIG. 23 is a diagram illustrating a configuration example of a vibration detecting apparatus according to a fifth embodiment;

FIG. 24 is a block diagram illustrating a configuration example of an acoustic processing unit and a display unit constituting a vibration detecting apparatus;

FIG. 25 is a block diagram illustrating a processing sequence of a display device;

FIG. 26 is a block diagram illustrating another configuration example of an acoustic processing unit and a display unit constituting a vibration detecting apparatus;

FIG. 27 is a flowchart illustrating a processing sequence of a display device;

FIG. 28 is a diagram illustrating a configuration example of a vibration detecting apparatus according to a sixth embodiment;

FIG. 29 is a diagram illustrating a configuration example of a vibration detecting apparatus according to a seventh embodiment;

FIG. 30 is a block diagram illustrating a configuration example of an acoustic processing unit and a headphone constituting a vibration detecting apparatus;

FIG. 31 is a block diagram illustrating a configuration example of a headphone that is configured such that an output state can be selectively switched;

FIG. 32 is a diagram illustrating a configuration example of a vibration detecting apparatus according to an eighth embodiment;

FIG. 33 is a block diagram illustrating a configuration example of an acoustic processing unit and a headphone constituting a vibration detecting apparatus;

FIG. 34 is a diagram illustrating a configuration example of a vibration detecting apparatus according to a ninth embodiment;

FIG. 35 is a diagram illustrating a configuration example of a vibration detecting apparatus according to a tenth embodiment;

FIG. 36 is a block diagram illustrating a configuration example of an acoustic processing unit and a headphone constituting a vibration detecting apparatus;

FIG. 37 is a diagram illustrating the case in which a correction filter included by a filter processing unit includes a static filter having a fixed filter characteristic and a dynamic filter having a variable filter characteristic;

FIG. 38 is a block diagram illustrating a configuration example of an acoustic processing unit and a headphone constituting a vibration detecting apparatus;

FIG. 39 is a sequence diagram illustrating an example of a communication sequence between a network communication unit of an acoustic processing unit and a server functioning as an external apparatus;

FIG. 40 is a diagram illustrating a configuration example of a vibration detecting apparatus according to an eleventh embodiment;

FIG. 41 is a block diagram illustrating a configuration example of an acoustic processing unit and a headphone constituting a vibration detecting apparatus;

FIG. 42 is a block diagram illustrating another configuration example of an acoustic processing unit and a headphone constituting a vibration detecting apparatus;

FIG. 43 is a diagram illustrating a configuration example of a vibration detecting apparatus according to a twelfth embodiment;

FIG. 44 is a block diagram illustrating a configuration example of an acoustic processing unit and a headphone constituting a vibration detecting apparatus;

FIG. 45 is a flowchart illustrating a processing sequence of an acoustic processing unit and a headphone;

FIG. 46 is a diagram illustrating a configuration example of a vibration detecting apparatus according to a thirteenth embodiment;

FIG. 47 is a block diagram illustrating a configuration example of a headphone constituting a vibration detecting apparatus;

FIG. 48 is a flowchart illustrating a processing sequence of a headphone;

FIG. 49 is a block diagram illustrating a configuration example of an electronic medical chart generating apparatus according to a fourteenth embodiment;

FIG. 50 is a block diagram illustrating a configuration example of a computer;

FIG. 51 is a block diagram illustrating a configuration example of a measurement supporting apparatus according to a fifteenth embodiment;

FIG. 52 is a diagram illustrating an example of measurement support information that is displayed on a display unit; and

FIG. 53 is a diagram illustrating a configuration example of an analog stethoscope according to the related art.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

The following description will be made in the order described below.

### 1. First Embodiment (Vibration Detecting Apparatus)

### 2. Second Embodiment (Vibration Detecting Apparatus)

### 3. Third Embodiment (Vibration Detecting Apparatus)

### 4. Fourth Embodiment (Vibration Detecting Apparatus)

### 5. Fifth Embodiment (Vibration Detecting Apparatus)

### 6. Sixth Embodiment (Vibration Detecting Apparatus)

### 7. Seventh Embodiment (Vibration Detecting Apparatus)

### 8. Eighth Embodiment (Vibration Detecting Apparatus)

### 9. Ninth Embodiment (Vibration Detecting Apparatus)

### 10. Tenth Embodiment (Vibration Detecting Apparatus)

### 11. Eleventh Embodiment (Vibration Detecting Apparatus)

### 12. Twelfth Embodiment (Vibration Detecting Apparatus)

### 13. Thirteenth Embodiment (Vibration Detecting Apparatus)

### 14. Fourteenth Embodiment (Electronic Medical Chart Generating Apparatus)

### 15. Fifteenth Embodiment (Measurement Supporting Apparatus)

### 16. Modification

### 1. First Embodiment

### Configuration Example of Vibration Detecting Apparatus

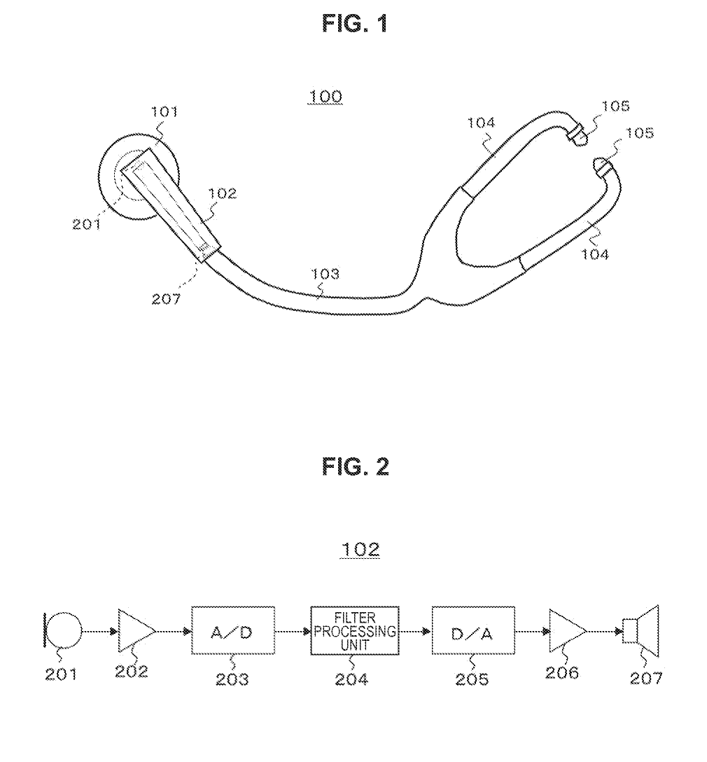

FIG. 1 illustrates a configuration example of a vibration detecting apparatus 100 according to a first embodiment. The vibration detecting apparatus 100 includes a chest piece 101, an acoustic processing unit 102, a rubber tube 103, ear tubes 104, and ear pieces 105.

The vibration detecting apparatus 100 has the same configuration as an analog stethoscope (refer to FIG. 53) according to the related art, except that the acoustic processing unit 102 is inserted between the chest piece 101 and the rubber tube 103. The acoustic processing unit 102 has a microphone and a speaker and performs correction of an acoustic characteristic (a frequency characteristic and a phase characteristic) with respect to a sound collected by the chest piece 101 and outputs the sound to the rubber tube 103. The sound propagates through the rubber tube 103 and the ear tube 104 and is guided from the ear piece 105 to an external auditory meatus of a user.

FIG. 2 illustrates a configuration example of the acoustic processing unit 102. The acoustic processing unit 102 has a microphone 201, an amplifier 202, an A/D converter 203, a filter processing unit 204, a D/A converter 205, an amplifier 206, and a speaker 207. The microphone 201 is mounted on the chest piece 101. The microphone 201 converts the sound (vibration) collected by the chest piece 101 into an acoustic signal (vibration signal) to be an electric signal. The microphone 201 constitutes a biological vibration detecting unit together with the chest piece 101.

The amplifier 202 amplifies the acoustic signal that is acquired by the microphone 201. The A/D converter 203 converts the acoustic signal output from the amplifier 202 from an analog signal to a digital signal. The filter processing unit 204 performs filtering to correct an acoustic characteristic (a frequency characteristic and a phase characteristic) with respect to the acoustic signal output from the A/D converter 203.

The filter processing unit 204 has a correction filter to perform filtering of a filter characteristic including an inverse characteristic of an entire acoustic characteristic or a partial acoustic characteristic of the chest piece 101, the rubber tube 103, the ear tube 104, the ear piece 105, the microphone 201, and the speaker 207. By the filtering, deterioration of a frequency characteristic in each portion such as the chest piece 101 can be compensated for and a standing wave of the rubber tube 103 or the ear tube 104 can be attenuated.

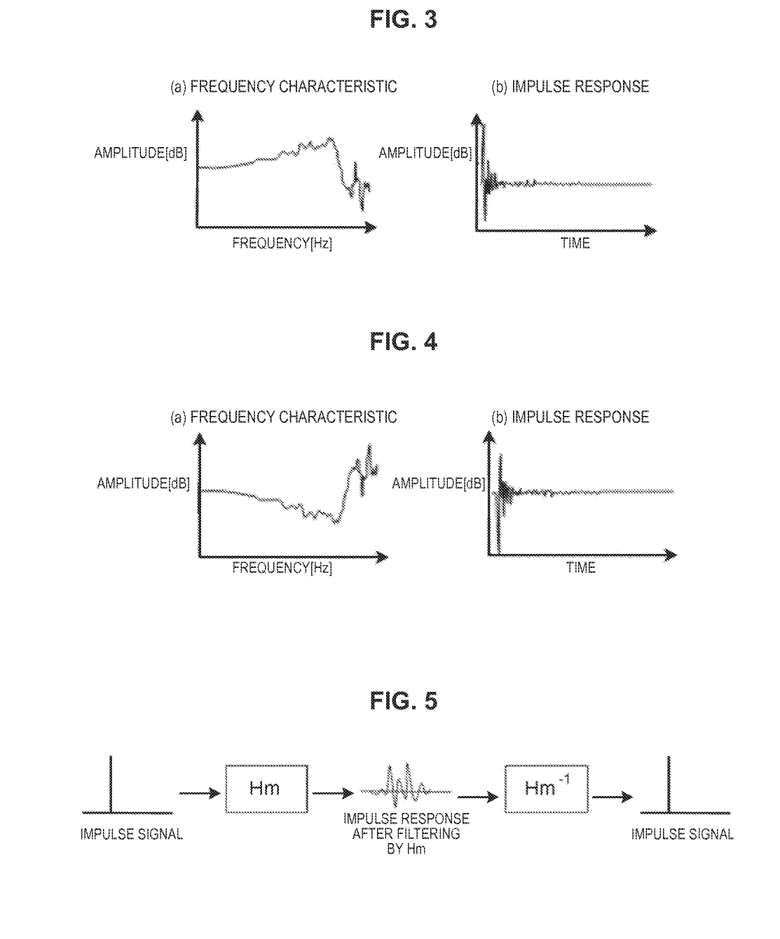

The filtering is performed by executing an impulse response convolution operation with respect to an acoustic signal. FIG. 3 illustrates an example of an acoustic characteristic Hm of the chest piece 101. FIG. 3(a) illustrates a frequency characteristic and FIG. 3(b) illustrates an impulse response. FIG. 4 illustrates an example of an inverse characteristic FIG. 4(a) illustrates a frequency characteristic and FIG. 4(b) illustrates an impulse response.

FIG. 5 illustrates a relation of an impulse signal, an impulse response after filtering by an acoustic characteristic Hm, and an impulse signal obtained by filtering the impulse response by a filter having an inverse characteristic Hm⁻¹. It can be seen from the relation that the acoustic signal of which the characteristic, has been deteriorated by the acoustic characteristic Hm is filtered with the inverse characteristic Hm⁻¹ and the acoustic signal can be corrected to flatten a frequency characteristic and allow a phase characteristic to be made into a linear phase.

The filter processing unit 204 may further have a correction filter that performs filtering to remove noise and a correction filer that performs filtering to convert an acoustic characteristic into a desired acoustic characteristic. Here, a filter having a constant group delay characteristic, for example, a finite impulse response (FIR) filter is used as the correction filter. In this case, the acoustic characteristic can be corrected without generating phase characteristic distortion.

The filter processing unit 204 has a single filter or a multi-stage filter as the correction filter. In the case of the multi-stage filter, the filter can include a predetermined number of static filters having a fixed filter configuration and a predetermined number of dynamic filters having a variable filter configuration.

The static filters include a correction filter to correct a characteristic of the ear piece of which the characteristic does not change or a correction filter to correct a characteristic of the chest piece 101 or the rubber tube 103 in a general use state. The dynamic filters include a correction filter to remove noise which varies according to a situation such as an environment or a correction filter to correct a characteristic change by a shape change of the chest piece 101 or the rubber tube 103 or a change of the pressing pressure of a diaphragm of the chest piece 101. The dynamic filters also include a correction filter to attenuate a standing wave of the rubber tube 103 or the ear tube 104 changed by a frequency of an acoustic signal.

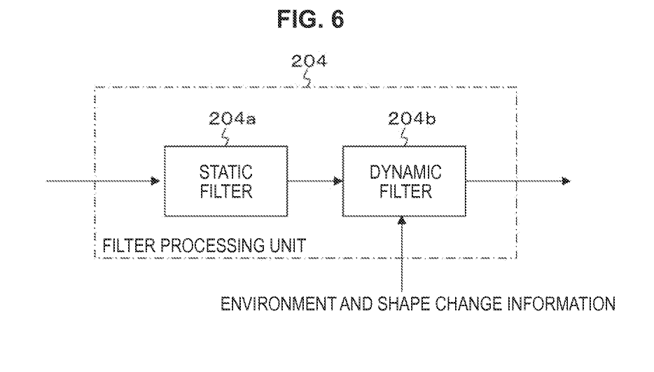

FIG. 6 illustrates an example of the case in which the filter processing unit 204 has a multi-stage filter as the correction filter. In this example, the case of two-stage configuration is illustrated and the filter processing unit 204 has a static filter 204a and a dynamic filter 204b. Environment information and shape change information are supplied to the dynamic filter 204b and a filter coefficient is switched according to an environment or shape change. In this case, the environment information and the shape change information are given by an input operation from the user or sensing by a sensor not illustrated in the drawings.

Returning to FIG. 2, the D/A converter 205 converts the acoustic signal output from the filter processing unit 204 from a digital signal to an analog signal. The amplifier 206 amplifies the acoustic signal that is output from the D/A converter 205. The speaker 207 outputs a sound obtained from the acoustic signal (vibration signal) output from the amplifier 206 to the rubber tube 103.

Next, an operation of the acoustic processing unit 102 illustrated in FIG. 2 will be described. In the microphone 201, a sound (vibration) that is collected by the chest piece 101 is converted into an acoustic signal (vibration signal) to be an electric signal. The acoustic signal is amplified by the amplifier 202, is converted by the A/D converter 203 from an analog signal to a digital signal, and is supplied to the filter processing unit 204.

In the filter processing unit 204, filtering of a filter characteristic including an inverse characteristic of an entire acoustic characteristic or a partial acoustic characteristic of the chest piece 101, the rubber tube 103, the ear tube 104, the ear piece 105, the microphone 201, and the speaker 207 is performed. By the filtering, deterioration of a frequency characteristic in each portion such as the chest piece 101 is compensated for and a standing wave of the rubber tube 103 or the ear tube 104 is attenuated. In the filter processing unit 204, filtering to remove noise and filtering to convert an acoustic characteristic into a desired acoustic characteristic may be performed.

The corrected acoustic signal output from the filter processing unit 204 is converted by the D/A converter 205 from a digital signal to an analog signal, is amplified by the amplifier 206, and is supplied to the speaker 207. A sound (vibration) that is obtained from the corrected acoustic signal is output from the speaker 207.

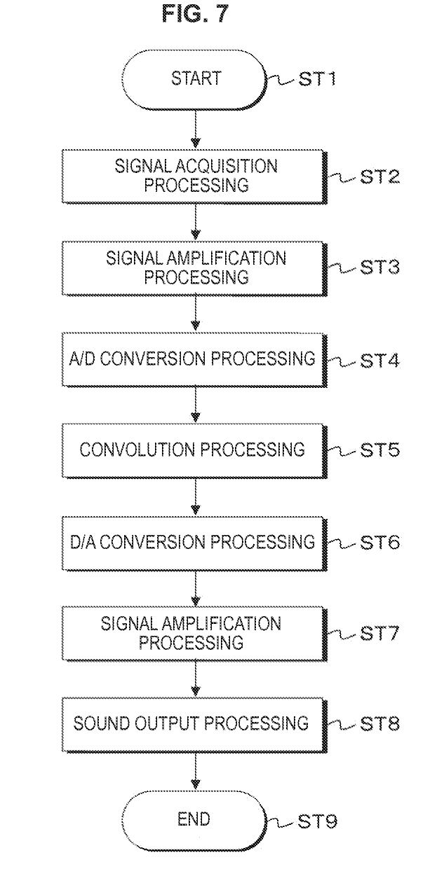

A flowchart of FIG. 7 illustrates a processing sequence of the acoustic processing unit 102 illustrated in FIG. 2. The acoustic processing unit 102 starts processing in step ST1 and proceeds to processing of step ST2. In step ST2, the acoustic processing unit 102 acquires an acoustic signal corresponding to a sound (vibration) collected by the chest piece 101, by the microphone 201.

Next, in step ST3, the acoustic processing unit 102 amplifies the acoustic signal acquired by the microphone 201. In step ST4, the acoustic processing unit 102 converts the amplified acoustic signal from an analog signal to a digital signal. In step ST5, the acoustic processing unit 102 convolutes a correction filter coefficient (impulse response) on the acoustic signal acquired by the microphone 201 and performs filtering of a correction filter characteristic.

Next, in step ST6, the acoustic processing unit 102 converts the corrected acoustic signal from a digital signal to an analog signal. In step ST7, the acoustic processing unit 102 amplifies the acoustic signal. In step ST8, the acoustic processing unit 102 outputs a sound (vibration) obtained from the corrected acoustic signal from the speaker 207. Then, in step ST9, the acoustic processing unit 102 ends the processing.

In the processing sequence illustrated in the flowchart of FIG. 7, the acoustic processing unit 102 executes convolution processing on a time axis in step ST5. However, the convolution processing may be executed on a frequency axis. When a tap length increases, the convolution processing is executed on the frequency axis, so that an operation amount can be decreased and an operation load can be alleviated.

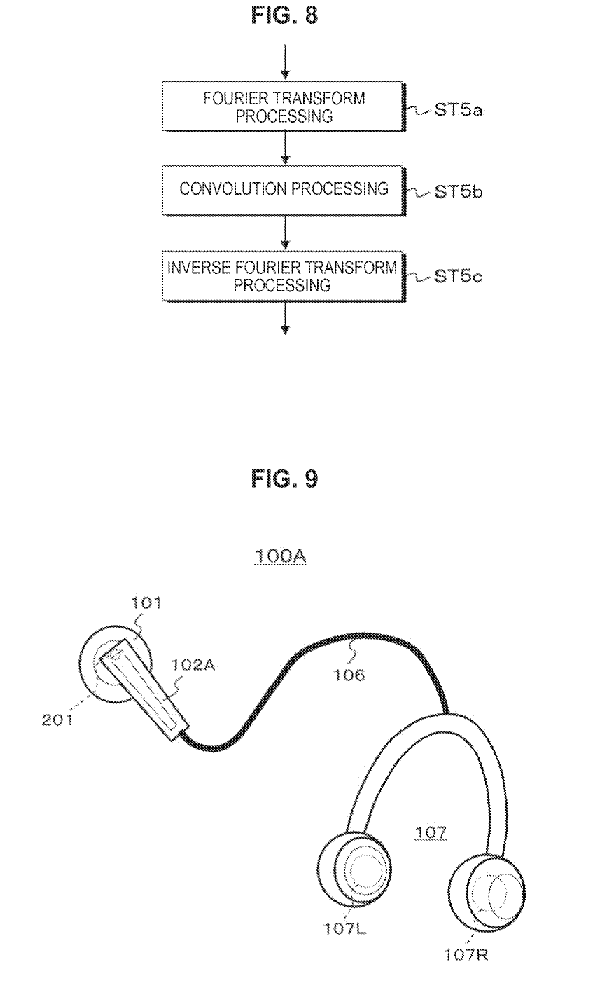

A flowchart of FIG. 8 illustrates an example of a processing sequence in the case in which the convolution processing is executed on the frequency axis. FIG. 8 illustrates only a portion corresponding to step ST5 of the flowchart of FIG. 7. In step ST5a, the acoustic processing unit 102 executes Fourier transform processing (FFT processing) for converting the acoustic signal acquired by the microphone 201 from signal data on the time axis to signal data on the frequency axis.

Next, in step ST5b, the acoustic processing unit 102 convolutes a correction filter coefficient (impulse response) on the signal data on the frequency axis and performs filtering of a correction filter characteristic. In step ST5c, the acoustic processing unit 102 executes inverse Fourier transform processing (IFFT processing) for converting the acoustic signal from signal data on the frequency axis to signal data on the time axis.

As described above, in the vibration detecting apparatus 100 illustrated in FIG. 1 filtering of a filter characteristic including an inverse characteristic of an entire acoustic characteristic or a partial acoustic characteristic of the chest piece 101, the rubber tube 103, the ear tube 104, the ear piece 105, the microphone 201, and the speaker 207 is executed by the acoustic processing unit 102. For this reason, deterioration of a frequency characteristic in each portion such as the chest piece 101 can be compensated for, a standing wave of the rubber tube 103 or the ear tube 104 can be attenuated, and a user can hear a biological vibration sound such as a cardiac sound or a pulmonary sound with a superior characteristic.

### 7. Second Embodiment

### Configuration Example of Vibration Detecting Apparatus

FIG. 9 illustrates a configuration example of a vibration detecting apparatus 100A according to a second embodiment. In FIG. 9, structural elements that correspond to the structural elements of FIG. 1 are denoted with the same reference numerals and repeated explanation of these structural elements is omitted. The vibration detecting apparatus 100A includes a chest piece 101, an acoustic processing unit 102A, a connection line 106, and a headphone 107.

The vibration detecting apparatus 100A has a shape significantly different from the shape of the analog stethoscope (refer to FIG. 53) according to the related art and does not include the rubber tube, the ear tube, and the ear piece. The vibration detecting apparatus 100A has a configuration in which the headphone 107 is connected to the acoustic processing unit 102A to which the chest piece 101 is connected, through the connection line 106. The acoustic processing unit 102A has one microphone 201 that is mounted on the chest piece 101. Correction of an acoustic characteristic (a frequency characteristic and a phase characteristic) is performed with respect to an acoustic signal (vibration signal) obtained by the microphone 201 and the corrected acoustic signal is supplied to the headphone 107 that a user wears, through the connection line 106.

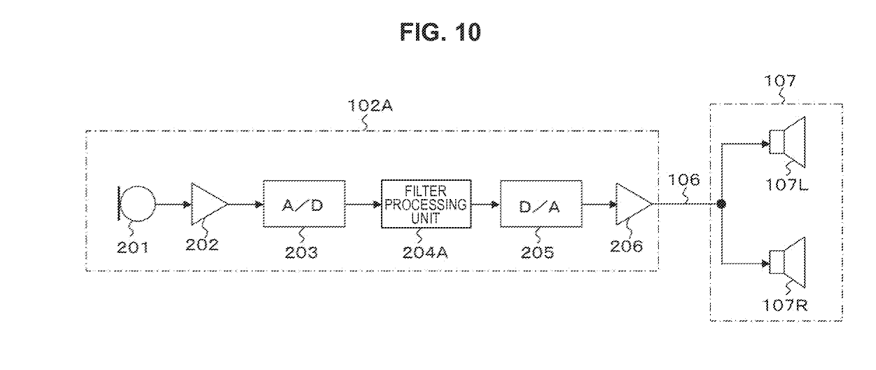

FIG. 10 illustrates a configuration example of the acoustic processing unit 102A and the headphone 107. In FIG. 10, structural elements that correspond to the structural elements of FIG. 2 are denoted with the same reference numerals and repeated explanation of these structural elements is omitted. The acoustic processing unit 102A has a microphone 201, an amplifier 202, an A/D converter 203, a filter processing unit 204A, a D/A converter 205, and an amplifier 206.

The microphone 201 is mounted on the chest piece 101. The microphone 201 converts a sound (vibration) collected by the chest piece 101 into an acoustic signal (vibration signal) to be an electric signal. The microphone 201 constitutes a biological vibration detecting unit together with the chest piece 101.

The amplifier 202 amplifies the acoustic signal that is acquired by the microphone 201. The A/D converter 203 converts the acoustic signal output from the amplifier 202 from an analog signal to a digital signal. The filter processing unit 204A performs filtering to correct an acoustic characteristic (a frequency characteristic and a phase characteristic) with respect to the acoustic signal output from the A/D converter 203.

The filter processing unit 204A has a correction filter to perform filtering of a filter characteristic including an inverse characteristic of an entire acoustic characteristic or a partial acoustic characteristic of the chest piece 101, the microphone 201, and speakers 107L and 107R. By the filtering, deterioration of a frequency characteristic in each portion such as the chest piece 101 can be compensated for. Similar to the filter processing unit 204 of the acoustic processing unit 102 of FIG. 2, the filtering is performed by executing an impulse response convolution operation with respect to an acoustic signal.

Similar to the filter processing unit 204 of the acoustic processing unit 102 of FIG. 2, the filter processing unit 204A may further have a correction filter that performs filtering to remove noise and a correction filer that performs filtering to convert an acoustic characteristic into a desired acoustic characteristic. Here, a filter having a constant group delay characteristic, for example, an FIR filter is used as the correction filter. Similar to the filter processing unit 204 of the acoustic processing unit 102 of FIG. 2, the filter processing unit 204A has a single filter or a multi-stage filter as the correction filter.

The D/A converter 205 converts the acoustic signal output from the filter processing unit 204A from a digital signal to an analog signal. The amplifier 206 amplifies the acoustic signal output from the D/A converter 205 and supplies the acoustic signal to the left speaker 107L and the right speaker 107R constituting the headphone 107, through the connection line 106.

Next, operations of the acoustic processing unit 102A and the headphone 107 illustrated in FIG. 10 will be described. In the microphone 201 of the acoustic processing unit 102A, a sound (vibration) that is collected by the chest piece 101 (refer to FIG. 9) is converted into an acoustic signal (vibration signal) to be an electric signal. The acoustic signal is amplified by the amplifier 202, is converted by the A/D converter 203 from an analog signal to a digital signal, and is supplied to the filter processing unit 204A.

In the filter processing unit 204A, filtering of a filter characteristic including an inverse characteristic of an entire acoustic characteristic or a partial acoustic characteristic of the chest piece 101, the microphone 201, and the speakers 107L and 107R is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is converted by the D/A converter 205 from a digital signal to an analog signal, is amplified by the amplifier 206, and is supplied to the left and right speakers 107L and 107R constituting the headphone 107, through the connection line 106. A sound (vibration) that is obtained from the corrected acoustic signal is output from the speakers 107L and 107R.

As described above, in the vibration detecting apparatus 100A illustrated in FIG. 9, filtering of a filter characteristic including an inverse characteristic of an entire acoustic characteristic or a partial acoustic characteristic of the chest piece 101, the microphone 201, and the speakers 107L and 107R is executed by the acoustic processing unit 102A. For this reason, deterioration of a frequency characteristic in each portion such as the chest piece 101 can be compensated for and a user can hear a biological vibration sound such as a cardiac sound or a pulmonary sound with a superior characteristic, using the headphone 107.

### 3. Third Embodiment

### Configuration Example of Vibration Detecting Apparatus

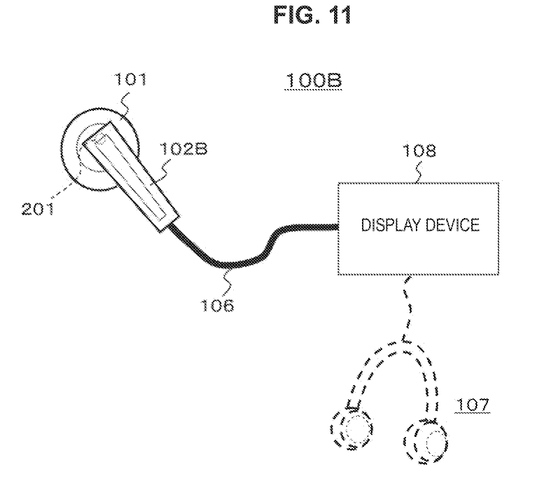

FIG. 11 illustrates a configuration example of a vibration detecting apparatus 100B according to a third embodiment. In FIG. 11, structural elements that correspond to the structural elements of FIGS. 1 and 9 are denoted with the same reference numerals and repeated explanation of these structural elements is omitted. The vibration detecting apparatus 100B includes a chest piece 101, an acoustic processing unit 102B, a connection line 106, and a display device 108.

The vibration detecting apparatus 100B has a configuration in which the display device 108 is connected to the acoustic processing unit 102B to which the chest piece 101 is connected, through the connection line 106. The acoustic processing unit 102B has a microphone 201 and performs correction of an acoustic characteristic (a frequency characteristic and a phase characteristic) with respect to a sound collected by the chest piece 101 and supplies the corrected acoustic signal to the display device 108 through the connection line 106. As the display device 108, a medical image display device and an image display device such as a smart phone or a tablet PC are considered.

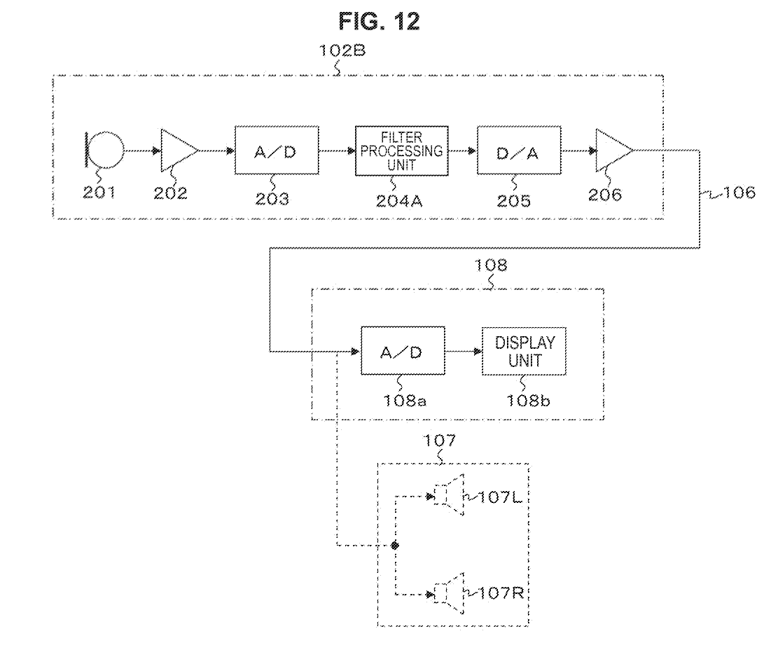

FIG. 12 illustrates a configuration example of the acoustic processing unit 102B and the display device 108. In FIG. 12, structural elements that correspond to the structural elements of FIG. 10 are denoted with the same reference numerals and repeated explanation of these structural elements is omitted. This example is an example of the case in which an acoustic signal (vibration signal) is transmitted as an analog signal from the acoustic processing unit 102B to the display device 108.

The acoustic processing unit 102B has the same configuration as the acoustic processing unit 102A illustrated in FIG. 10. That is, the acoustic processing unit 102B has a microphone 201, an amplifier 202, an A/D converter 203, a filter processing unit 204A, a D/A converter 205, and an amplifier 206. The amplifier 206 supplies the corrected acoustic signal to the display device 108 through the connection line 106

The display device 108 has an A/D converter 108a and a display unit 108b. The A/D converter 108a converts the acoustic signal transmitted from the acoustic processing unit 102B from an analog signal to a digital signal. The display unit 108b displays a waveform and/or a frequency spectrum, based on the acoustic signal converted into the digital signal. In this case, in the waveform or the frequency spectrum, a change corresponding to abnormality appears, when there is the abnormality in a biological vibration such as a cardiac sound or a pulmonary sound. For this reason, a doctor can diagnose illness, based on display of the waveform or the frequency spectrum.

Next, operations of the acoustic processing unit 102B and the display device 108 illustrated in FIG. 12 will be described. In the microphone 201, a sound (vibration) that is collected by the chest piece 101 is converted into an acoustic signal (vibration signal) to be an electric signal. The acoustic signal is amplified by the amplifier 202, is converted by the A/D converter 203 from an analog signal to a digital signal, and is supplied to the filter processing unit 204A.

In the filter processing unit 204A, filtering of a filter characteristic including an inverse characteristic of an entire acoustic characteristic or a partial acoustic characteristic of the chest piece 101 and the microphone 201 is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is converted by the D/A converter 205 from a digital signal to an analog signal, is amplified by the amplifier 206, and is supplied to the display device 108 through the connection line 106. In the display device 108, the acoustic signal that is supplied from the acoustic processing unit 1023 is converted by the A/D converter 108a from an analog signal to a digital signal and is supplied to the display unit 108b. The waveform and/or the frequency spectrum is displayed on the display unit 108b, based on the corrected acoustic signal.

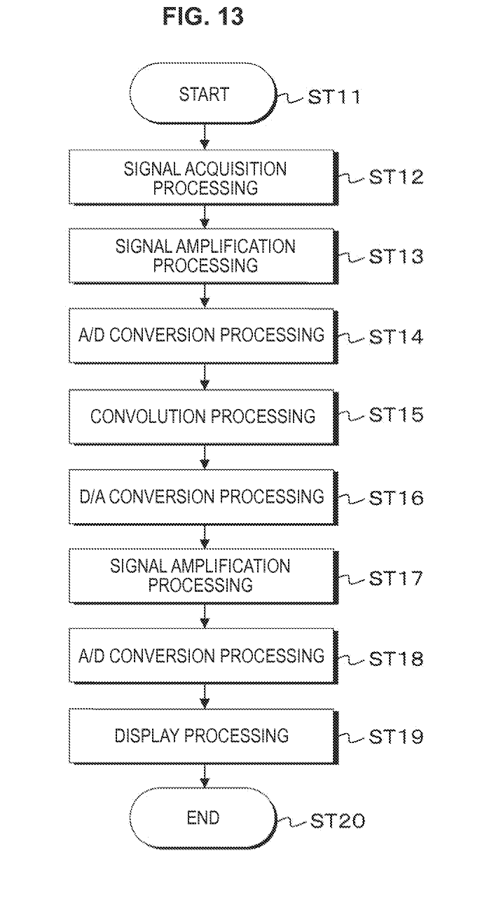

A flowchart of FIG. 13 illustrates a processing sequence of the acoustic processing unit 102B and the display device 108 illustrated in FIG. 12. The acoustic processing unit 102B and the display device 108 start processing in step ST11 and proceed to processing of step ST12. In step ST12, the acoustic processing unit 1023 acquires an acoustic signal corresponding to a sound (vibration) collected by the chest piece 101, by the microphone 201.

Next, in step ST13, the acoustic processing unit 102B amplifies the acoustic signal acquired by the microphone 201. In step ST14, the acoustic processing unit 102B converts the amplified acoustic signal from an analog signal to a digital signal. In step ST15, the acoustic processing unit 102B convolutes a correction filter coefficient (impulse response) on the acoustic signal acquired by the microphone 201 and performs filtering of a correction filter characteristic.

Next, in step ST16, the acoustic processing unit 102B converts the corrected acoustic signal from a digital signal to an analog signal. In step ST17, the acoustic processing unit 102B amplifies the acoustic signal and supplies the acoustic signal to the display device 108.

Next, in step ST18, the display device 108 converts the corrected acoustic signal supplied from the acoustic processing unit 102B front an analog signal to a digital signal. In step ST 19, the display device 108 displays a waveform and/or a frequency spectrum, based on the corrected acoustic signal. Then, in step ST20, the acoustic processing unit 102B and the display device 108 end the processing.

In the processing sequence illustrated in the flowchart of FIG. 13, the acoustic processing unit 102B executes convolution processing on a time axis in step ST15. Although detailed explanation is omitted, the convolution processing may be executed on the frequency axis. When a tap length increases, the convolution processing is executed on the frequency axis, so that an operation amount can be decreased and an operation load can be alleviated.

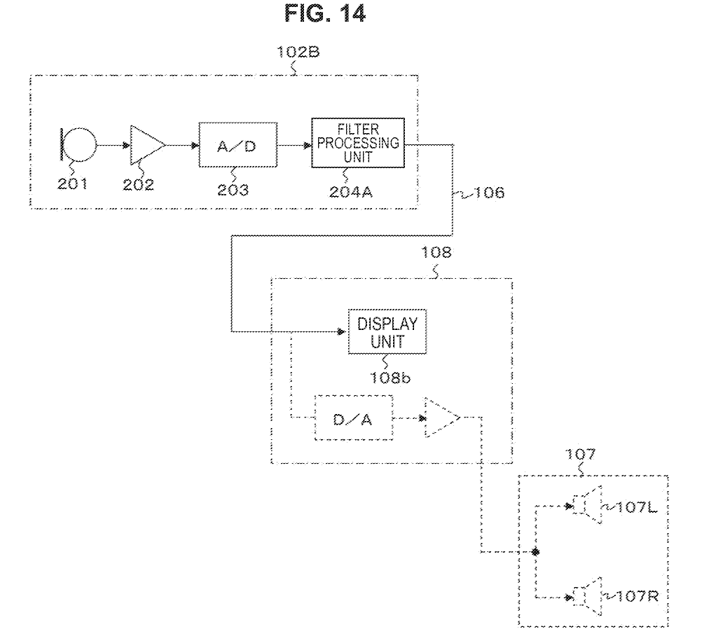

FIG. 14 illustrates another configuration example of the acoustic processing unit 102B and the display device 108. In FIG. 14, structural elements that correspond to the structural elements of FIGS. 10 and 12 are denoted with the same reference numerals and repeated explanation of these structural elements is omitted. This example is an example of the case in which an acoustic signal (vibration signal) is transmitted as a digital signal from the acoustic processing unit 102B to the display device 108.

The acoustic processing unit 102B has the configuration in which the D/A converter 205 and the amplifier 206 are removed from the acoustic processing unit 102A illustrated in FIG. 10. That is, the acoustic processing unit 102B has a microphone 201, an amplifier 202, an A/D converter 203, and a filter processing unit 204A. The filter processing unit 204A supplies the corrected acoustic signal to the display device 108 through the connection line 106.

The display device 108 has a display unit 108b. The display unit 108b displays a waveform and/or a frequency spectrum, based on the acoustic signal supplied from the acoustic processing unit 102B. In this case, in the waveform or the frequency spectrum, a change corresponding to abnormality appears, when there is the abnormality in a biological vibration such as a cardiac sound or a pulmonary sound. For this reason, a doctor can diagnose illness, based on display of the waveform or the frequency spectrum.

Next, operations of the acoustic processing unit 1023 and the display device 108 illustrated in FIG. 14 will be described. In the microphone 201, a sound 30(vibration) that is collected by the chest piece 101 is converted into an acoustic signal (vibration signal) to be an electric signal. The acoustic signal is amplified by the amplifier 202, is converted by the A/D converter 203 from an analog signal to a digital signal, and is supplied to the filter processing unit 204A.

In the filter processing unit 204A, filtering of a filter characteristic including an inverse characteristic of an entire acoustic characteristic or a partial acoustic characteristic of the chest piece 101 and the microphone 201 is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is supplied to the display device 108 through the connection line 106. In the display device 108, the corrected acoustic signal that is supplied from the acoustic processing unit 102B is supplied to the display unit 108b. The waveform and/or the frequency spectrum is displayed on the display unit 108b, based on the corrected acoustic signal.

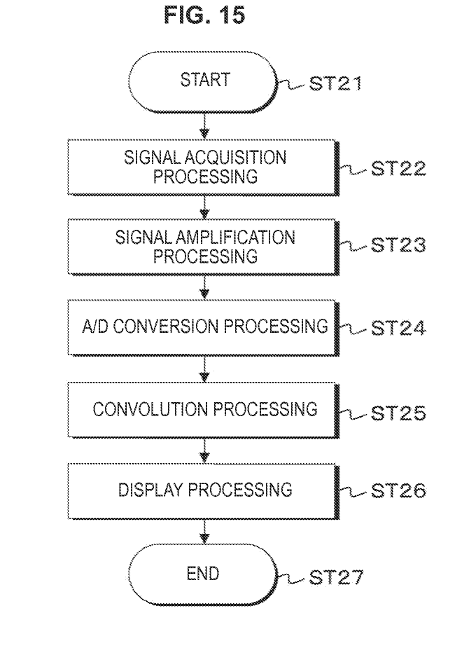

A flowchart of FIG. 15 illustrates a processing sequence of the acoustic processing unit 102B and the display device 108 illustrated in FIG. 14. The acoustic processing unit 1023 and the display device 108 start processing in step ST21 and proceed to processing of step ST22. In step ST22, the acoustic processing unit 102B acquires an acoustic signal corresponding to a sound (vibration) collected by the chest piece 101, by the microphone 201.

Next, in step ST23, the acoustic processing unit 1023 amplifies the acoustic signal acquired by the microphone 201. In step ST24, the acoustic processing unit 1023 converts the amplified acoustic signal from an analog signal to a digital signal. In step ST25, the acoustic processing unit 1023 convolutes a correction filter coefficient (impulse response) on the acoustic signal acquired by the microphone 201, performs filtering of a correction filter characteristic, and supplies the acoustic signal to the display device 108.

Next, in step ST26, the display device 108 displays the waveform and/or the frequency spectrum, based on the corrected acoustic signal supplied from the acoustic processing unit 102B. Then, in step ST27, the acoustic processing unit 102B and the display device 108 end the processing.

In the processing sequence illustrated in the flowchart of FIG. 15, the acoustic processing unit 102B executes convolution processing on a time axis in step ST25. Although detailed explanation is omitted, the convolution processing may be executed on the frequency axis. When a tap length increases, the convolution processing is executed on the frequency axis, so that an operation amount can be decreased and an operation load can be alleviated.

As described above, in the vibration detecting apparatus 100B illustrated in FIG. 11, filtering of a filter characteristic including an inverse characteristic of an entire acoustic characteristic or a partial acoustic characteristic of the chest piece 101 and the microphone 201 is executed by the acoustic processing unit 102B. For this reason, deterioration of a frequency characteristic in each portion such as the chest piece 101 can be compensated for and a user can observe a waveform or a frequency spectrum of a biological vibration sound such as a cardiac sound or a pulmonary sound with a superior characteristic, using the display device 108.

As described above, in the vibration detecting apparatus 100B that displays the waveform and/or frequency spectrum of the corrected acoustic signal on the display device 108, as shown by a broken line in FIG. 11, the headphone 107 is connected to the display device 108, so that the user can hear a sound obtained from the corrected acoustic signal, that is, a biological vibration sound such as a cardiac sound or a pulmonary sound with a superior characteristic. In this case, for the headphone 107, the display device 108 constitutes a repeater.

In the case of the analog transmission configuration illustrated in FIG. 12, as shown by a broken line, the acoustic signal that is supplied from the acoustic processing unit 102B to the display device 108 is supplied to the speakers 107L and 107R constituting the headphone 107. In the case of the digital transmission configuration illustrated in FIG. 14, as shown by a broken line, the acoustic signal that is supplied from the acoustic processing unit 102B to the display device 108 is converted by the D/A converter from a digital signal to an analog signal, is amplified by the amplifier, and is supplied to the speakers 107L and 107R constituting the headphone 107.

In the configuration examples illustrated in FIGS. 12 and 14, the acoustic processing unit 102B has the filter processing unit 204A and the filtering processing of the correction filter is executed by the acoustic processing unit 102B. Although detailed explanation is omitted, the display device 108 may have the filter processing unit 204A and the filtering processing of the correction filter may be executed by the display device 108. In this case, the display device 108 can selectively apply the correction filter and other signal processing.

### 2. Fourth Embodiment

### Configuration Example of Vibration Detecting Apparatus

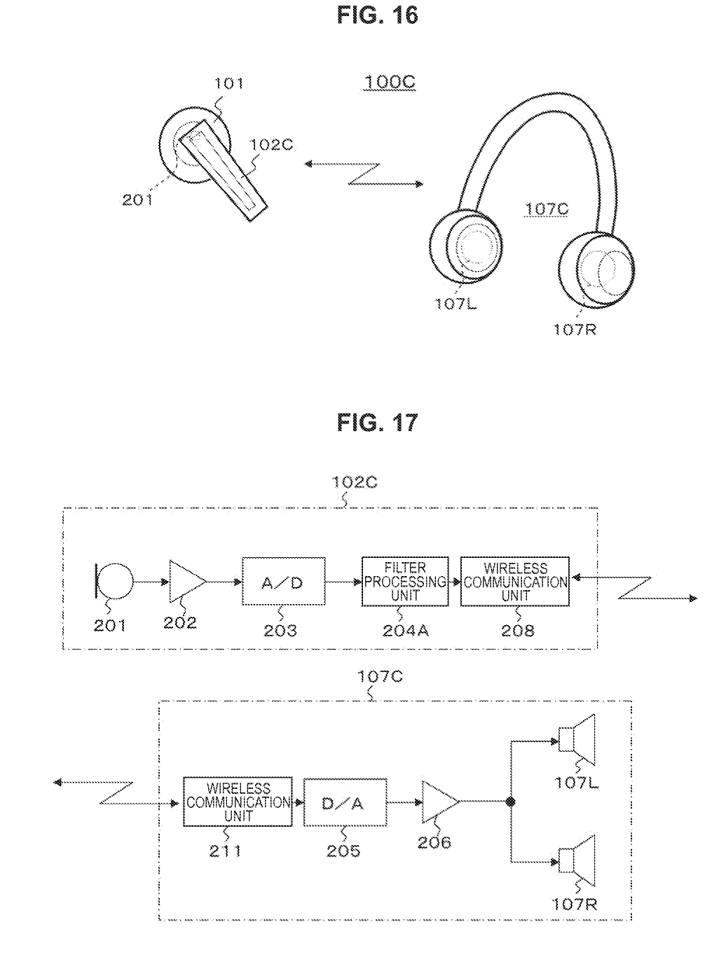

FIG. 16 illustrates a configuration example of a vibration detecting apparatus 100C according to a fourth embodiment. In FIG. 16, structural elements that correspond to the structural elements of FIGS. 1 and 9 are denoted with the same reference numerals and repeated explanation of these structural elements is omitted. The vibration detecting apparatus 100C includes a chest piece 101, an acoustic processing unit 102C, and a headphone 107C.

The vibration detecting apparatus 100C has a configuration in which the headphone 107C is wirelessly connected to the acoustic processing unit 102C to which the chest piece 101 is connected, through the connection line; 106. The acoustic processing unit 102C has a microphone 201 and performs correction of an acoustic characteristic (a frequency characteristic and a phase characteristic) with respect to a sound collected by the chest piece 101 and wirelessly transmits the corrected acoustic signal to the headphone 107C that a user wears.

For example, near field communication such as Bluetooth is used as wireless communication. The headphone 107C executes authentication processing for wireless connection between the acoustic processing unit 102C and the headphone 107C, according to a user operation or automatically, if necessary, and enters a wireless connection state. For example, in the case of the Bluetooth, pairing is performed or released based on the user operation.

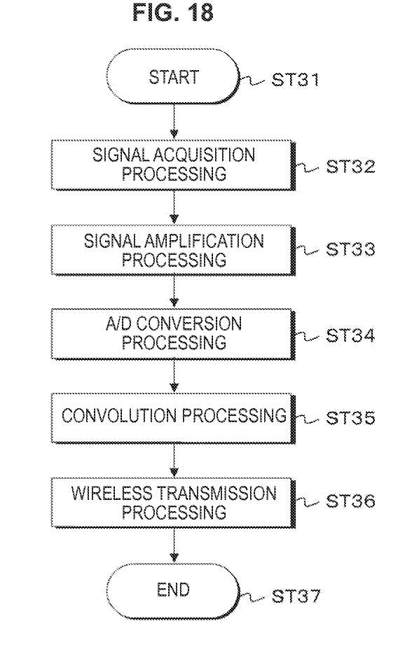

FIG. 17 illustrates a configuration example of the acoustic processing unit 102C and the headphone 107C. In FIG. 17, structural elements that correspond to the structural elements of FIG. 10 are denoted with the same reference numerals and repeated explanation of these structural elements is omitted. The acoustic processing unit 102C has a microphone 201, an amplifier 202, an A/D converter 203, a filter processing unit 204A, and a wireless communication unit 208.

The microphone 201 is mounted on the chest piece 101. The microphone 201 converts a sound (vibration) collected by the chest piece 101 into an acoustic signal (vibration signal) to be an electric signal. The microphone 201 constitutes a biological vibration detecting unit together with the chest piece 101.

The amplifier 202 amplifies the acoustic signal that is acquired by the microphone 201. The A/D converter 203 converts the acoustic signal output from the amplifier 202 from an analog signal to a digital signal. The filter processing unit 204A performs filtering to correct an acoustic characteristic (a frequency characteristic and a phase characteristic) with respect to the acoustic signal output from the A/D converter 203.

The filter processing unit 204A has a correction filter to perform filtering of a filter characteristic including an inverse characteristic of an entire acoustic characteristic or a partial acoustic characteristic of the chest piece 101, the microphone 201, and speakers 107L and 107R. By the filtering, deterioration of a frequency characteristic in each portion such as the chest piece 101 can be compensated for.

The wireless communication unit 208 performs communication between the headphone 107C and the wireless communication unit 208. That is, the wireless communication unit 208 transmits the corrected acoustic signal (vibration signal) output from the filter processing unit 204A to the headphone 107C. The wireless communication unit 208 performs communication for wireless connection processing between the headphone 107C and the wireless communication unit 208.

The headphone 107C has a wireless communication unit 211, a D/A converter 205, an amplifier 206, a left speaker 107L and a right speaker 107R. The wireless communication unit 211 performs communication between the acoustic processing unit 102C and the wireless communication unit 211. That is, the wireless communication unit 211 receives the corrected acoustic signal (vibration signal) that is transmitted from the acoustic processing unit 102C. The wireless communication unit 211 performs communication for wireless connection processing between the acoustic processing unit 102C and the wireless communication unit 211.

The D/A converter 205 converts the acoustic signal received by the wireless communication unit 211 from a digital signal to an analog signal. The amplifier 206 amplifies the acoustic signal output from the D/A converter 205 and supplies the acoustic signal to the left speaker 107L and the right speaker 107R.

Next, operations of the acoustic processing unit 102C and the headphone 107C illustrated in FIG. 17 will be described. In the microphone 201 of the acoustic processing unit 102C, a sound (vibration) that is collected by the chest piece 101 (refer to FIG. 16) is converted into an acoustic signal (vibration signal) to be an electric signal. The acoustic signal is amplified by the amplifier 202, is converted by the A/D converter 203 from an analog signal to a digital signal, and is supplied to the filter processing unit 204A.

In the filter processing unit 204A, filtering of a filter characteristic including an inverse characteristic of an entire acoustic characteristic or a partial acoustic characteristic of the chest piece 101 and the microphone 201 is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is supplied to the wireless communication unit 208. The corrected acoustic signal is wirelessly transmitted from the wireless communication unit 208 to the headphone 107C.

In the wireless communication unit 211 of the headphone 107C, the corrected acoustic signal (vibration signal) that is transmitted from the acoustic processing unit 102C is received. The acoustic signal is converted by the D/A converter 205 from a digital signal to an analog signal, is amplified by the amplifier 206, and is supplied to the left and right speakers 107L and 107R. A sound (vibration) that is obtained from the corrected acoustic signal is output to these speakers 107L and 107R.

A flowchart of FIG. 18 illustrates a processing sequence of the acoustic processing unit 102C illustrated in FIG. 17. The acoustic processing unit 102C starts processing in step ST31 and proceeds to processing of step ST32. In step ST32, the acoustic processing unit 102C acquires an acoustic signal corresponding to a sound (vibration) collected by the chest piece 101, by the microphone 201.

Next step ST33, the acoustic processing unit 102C amplifies the acoustic signal acquired by the microphone 201. In step ST34, the acoustic processing unit 102C converts the amplified acoustic signal from an analog signal to a digital signal. In step ST35, the acoustic processing unit 102C convolutes a correction filter coefficient (impulse response) on the acoustic signal acquired by the microphone 201 and performs filtering of a correction filter characteristic.

Next, in step ST36, the acoustic processing unit 102C wirelessly transmits the corrected acoustic signal obtained by step ST35 from the wireless communication unit 208 to the headphone 107C. Then, in step ST37, the acoustic processing unit 102C ends the processing.

In the processing sequence illustrated in the flowchart of FIG. 18, the acoustic processing unit 102C executes convolution processing on a time axis in step ST35. However, the convolution processing may be executed on the frequency axis. When a tap length increases, the convolution processing is executed on the frequency axis, so that an operation amount can be decreased and an operation load can be alleviated.

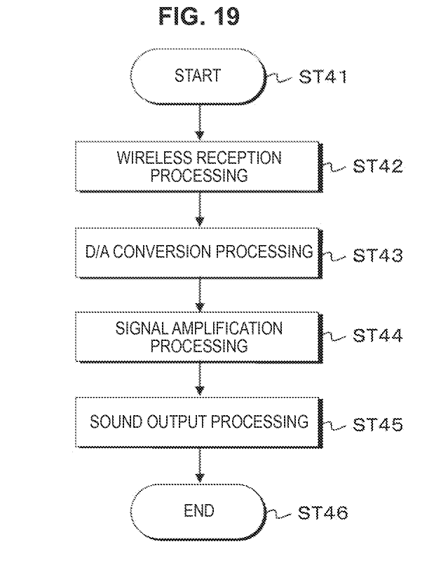

A flowchart of FIG. 19 illustrates a processing sequence of the headphone 107C illustrated in FIG. 17. The headphone 107C starts processing in step ST41 and proceeds to processing of step ST42. In step ST42, the headphone 107C receives the corrected acoustic signal (vibration signal) transmitted from the acoustic processing unit 102C, by the wireless communication unit 211.

Next, in step ST43, the headphone 107C converts the received corrected acoustic signal from a digital signal to an analog signal. In step ST44, the headphone 107C amplifies the acoustic signal. In step ST45, the headphone 107C outputs a sound (vibration) obtained from the corrected acoustic signal from the speakers 107L and 107R. Then, in step ST46, the headphone 107C ends the processing.

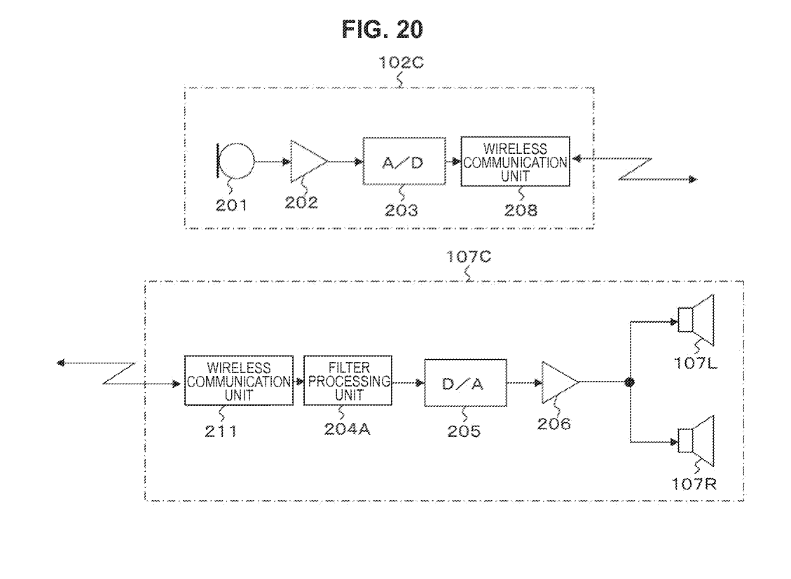

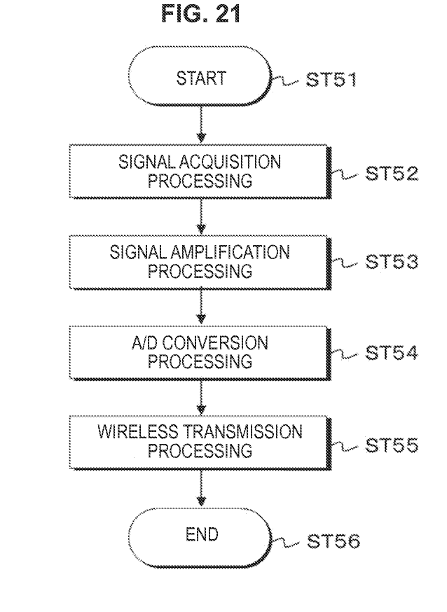

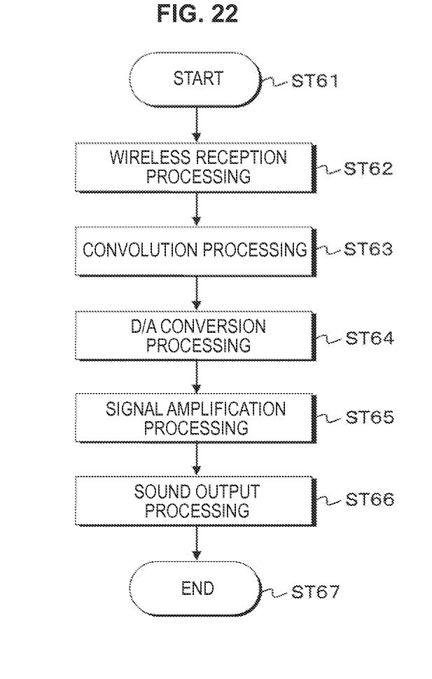

FIG. 20 illustrates another configuration example of the acoustic processing unit 102C and the headphone 107C. In this example, the headphone 107C has the filter processing unit 204A. In FIG. 20, structural elements that correspond to the structural elements of FIG. 17 are denoted with the same reference numerals and repeated explanation of these structural elements is omitted. The acoustic processing unit 102C has a microphone 201, an amplifier 202, an A/D converter 203, and a wireless communication unit 208.

The microphone 201 is mounted on the chest piece 101. The microphone 201 converts a sound (vibration) collected by the chest piece 101 into an acoustic signal (vibration signal) to be an electric signal. The microphone 201 constitutes a biological vibration detecting unit together with the chest piece 101.

The amplifier 202 amplifies the acoustic signal that is acquired by the microphone 201. The A/D converter 203 converts the acoustic signal output from the amplifier 202 from an analog signal to a digital signal. The wireless communication unit 208 performs communication between the headphone 107C and the wireless communication unit 208. That is, the wireless communication unit 208 transmits the acoustic signal (vibration signal) amplified by the amplifier 203 to the headphone 107C. The wireless communication unit 208 performs communication for wireless connection processing between the headphone 107C and the wireless communication unit 208.

The headphone 107C has a wireless communication unit 211, a filter processing unit 204A, a D/A converter 205, an amplifier 206, a left speaker 107L, and a right speaker 107R. The wireless communication unit 211 performs communication between the acoustic processing unit 102C and the wireless communication unit 211. That is, the wireless communication unit 211 receives the acoustic signal (vibration signal) that is transmitted from the acoustic processing unit 102C. The wireless communication unit 211 performs communication for wireless connection processing between the acoustic processing unit 102C and the wireless communication unit 211, at the time of wireless connection with the acoustic processing unit 102C.

The filter processing unit 204A has a correction filter to perform filtering of a filter characteristic including an inverse characteristic of an entire acoustic characteristic or a partial acoustic characteristic of the chest piece 101, the microphone 201, and the speakers 107L and 107R. By the filtering, deterioration of a frequency characteristic in each portion such as the chest piece 101 can be compensated for.

The D/A converter 205 converts the corrected acoustic signal (vibration signal) output from the filter processing unit 204A from a digital signal to an analog signal. The amplifier 206 amplifies the acoustic signal output from the D/A converter 205 and supplies the acoustic signal to the left speaker 107L and the right speaker 107R.

Next, operations of the acoustic processing unit 102C and the headphone 107C illustrated in FIG. 20 will be described. In the microphone 201 of the acoustic processing unit 102C, a sound (vibration) that is collected by the chest piece 101 (refer to FIG. 16) is converted into an acoustic signal (vibration signal) to be an electric signal. The acoustic signal is amplified by the amplifier 202 and is converted by the AD converter 203 from an analog signal to a digital signal. The acoustic signal is transmitted from the wireless communication unit 208 to the headphone 107C.

In the wireless communication unit 211 of the headphone 107C, the acoustic signal (vibration signal) that is transmitted from the acoustic processing unit 102C is received. The acoustic signal is supplied to the filter processing unit 204A. In the filter processing unit 204A, filtering of a filter characteristic including an inverse characteristic of an entire acoustic characteristic or a partial acoustic characteristic of the chest piece 101, the microphone 201, and the speakers 107L and 107R is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is converted by the D/A converter 205 from a digital signal to an analog signal, is amplified by the amplifier 206, and is supplied to the left and right speakers 107L and 107R. A sound (vibration) that is obtained from the corrected acoustic signal is output from these speakers 107L and 107R.

A flowchart of FIG. 21 illustrates a processing sequence of the acoustic processing unit 102C illustrated in FIG. 20. The acoustic processing unit 102C starts processing in step ST51 and proceeds to processing of step ST52. In step ST52, the acoustic processing unit 102C acquires an acoustic signal corresponding to a sound (vibration) collected by the chest piece 101, by the microphone 201.

Next, in step ST53, the acoustic processing unit 102C amplifies the acoustic signal acquired by the microphone 201. In step ST54, the acoustic processing unit 102C converts the amplified acoustic signal from an analog signal to a digital signal. In step ST55, the acoustic processing unit 102C wirelessly transmits the acoustic signal amplified by step ST53 from the wireless communication unit 208 to the headphone 107C. Then, in step ST56, the acoustic processing unit 102C ends the processing.

A flowchart of FIG. 22 illustrates a processing sequence of the headphone 107C illustrated in FIG. 20. The headphone 107C starts processing in step ST61 and proceeds to processing of step ST62. In step ST62, the headphone 107C receives the acoustic signal (vibration signal) transmitted from the acoustic processing unit 102C, by the wireless communication unit 211.

Next, in step ST63, the headphone 107C convolutes a correction filter coefficient (impulse response) on the received acoustic signal and performs filtering of a correction filter characteristic. In step ST64, the headphone 107C converts the corrected acoustic signal from a digital signal to an analog signal. In step ST65, the headphone 107C amplifies the acoustic signal. In step ST66, the headphone 107C outputs a sound (vibration) obtained from the corrected acoustic signal from the speakers 107L and 107R. Then, in step ST67, the headphone 107C ends the processing.

In the processing sequence illustrated in the flowchart of FIG. 22, the headphone 107C acoustic processing unit 102 executes convolution processing on a time axis in step ST63. However, the convolution processing may be executed on a frequency axis. When a tap length increases, the convolution processing is executed on the frequency axis, so that an operation amount can be decreased and an operation load can be alleviated.

As described above, in the vibration detecting apparatus 100C illustrated in FIG. 16, filtering of a filter characteristic including an inverse characteristic of an entire acoustic characteristic or a partial acoustic characteristic of the chest piece 101, the microphone 201, and the speakers 107L and 107R is executed by the acoustic processing unit 102C. For this reason, deterioration of a frequency characteristic in each portion such as the chest piece 101 can be compensated for and a user can hear a biological vibration sound such as a cardiac sound or a pulmonary sound with a superior characteristic, using the headphone 107C.

### 5. Fifth Embodiment

### Configuration Example of Vibration Detecting Apparatus

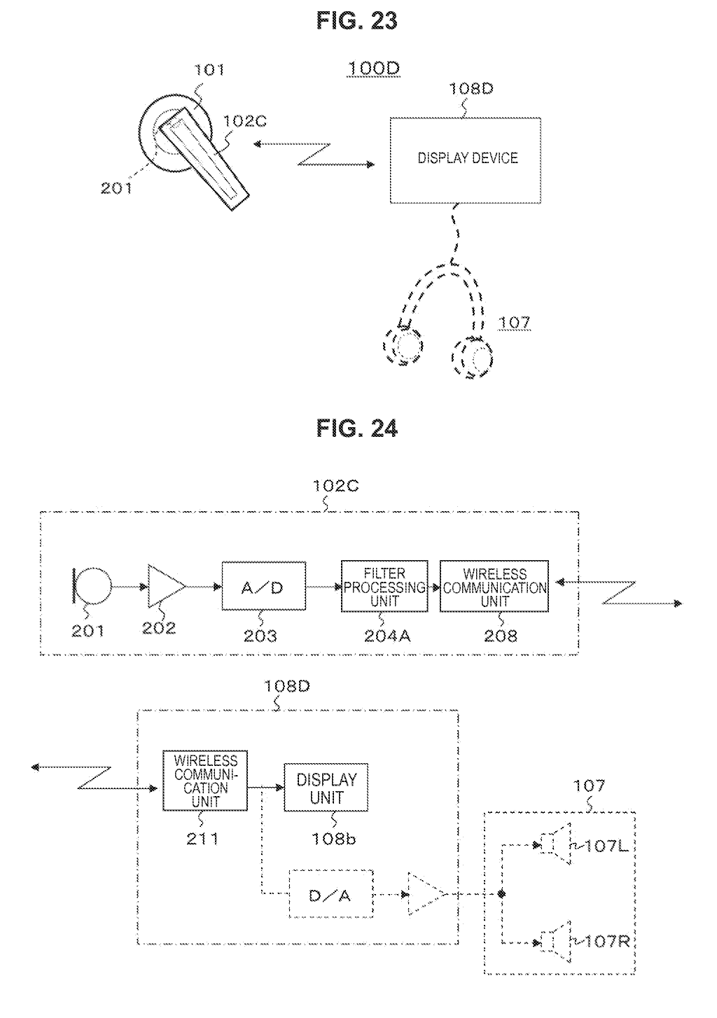

FIG. 23 illustrates a configuration example of a vibration detecting apparatus 100D according to a fifth embodiment. In FIG. 23, structural elements that correspond to the structural elements of FIGS. 11 and 16 are denoted with the same reference numerals and repeated explanation of these structural elements is omitted. The vibration detecting apparatus 100D includes a chest piece 101, an acoustic processing unit 102C, and a display device 108D.

The vibration detecting apparatus 100D has a configuration in which the display device 108D is wirelessly connected to the acoustic processing unit 102C to which the chest piece 101 is connected. The acoustic processing unit 102C has the same configuration as the acoustic processing unit 102C in the vibration detecting apparatus 100C illustrated in FIG. 16 described above. The acoustic processing unit 102C has a microphone 201 and performs correction of an acoustic characteristic (a frequency characteristic and a phase characteristic) with respect to a sound collected by the chest piece 101 and wirelessly transmits the corrected acoustic signal to the display device 108D.

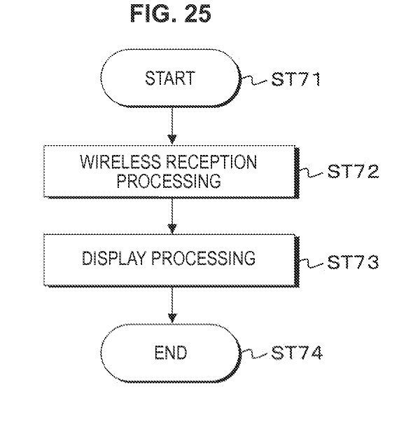

FIG. 24 illustrates a configuration example of the acoustic processing unit 102C and the display device 108D. In FIG. 24, structural elements that correspond to the structural elements of FIGS. 11 and 17 are denoted with the same reference numerals and repeated explanation of these structural elements is omitted. Similar to the acoustic processing unit 102C of FIG. 17, the acoustic processing unit 102C has a microphone 201, an amplifier 202, an A/D converter 203, a filter processing unit 204A, and a wireless communication unit 208.

The display device 108D has a wireless communication unit 211 and a display unit 108b. The wireless communication unit 211 performs communication between the acoustic processing unit 102C and the wireless communication unit 211. That is, the wireless communication unit 211 receives the corrected acoustic signal (vibration signal) that is transmitted from the acoustic processing unit 102C. The wireless communication unit 211 performs communication for wireless connection processing between the acoustic processing unit 102C and the wireless communication unit 211. The display unit 108b displays a waveform and/or a frequency spectrum, based on the received corrected acoustic signal.

Next, operations of the acoustic processing unit 102C and the display device 108D illustrated in FIG. 24 will be described. The corrected acoustic signal that is output from the filter processing unit 204A of the acoustic processing unit 102C is supplied to the wireless communication unit 208. The corrected acoustic signal (vibration signal) is wirelessly transmitted from the wireless communication unit 208 to the display device 108D.

In the wireless communication unit 211 of the display device 108D, the corrected acoustic signal (vibration signal) that is transmitted from the acoustic processing unit 102C is received. The acoustic signal is supplied to the display unit 108b. The waveform and/or the frequency spectrum is displayed on the display unit 108b, based on the corrected acoustic signal.

A flowchart of FIG. 25 illustrates a processing sequence of the display device 108D illustrated in FIG. 24. The display device 108D starts processing in step ST71 and proceeds to processing of step ST72. In step ST72, the display device 108D receives the corrected acoustic signal (vibration signal) transmitted from the acoustic processing unit 102C, by the wireless communication unit 211.

Next, in step ST73, the display device 108D displays the waveform and/or the frequency spectrum, based on the received corrected acoustic signal. Then, in step ST74, the display device 108D ends the processing.

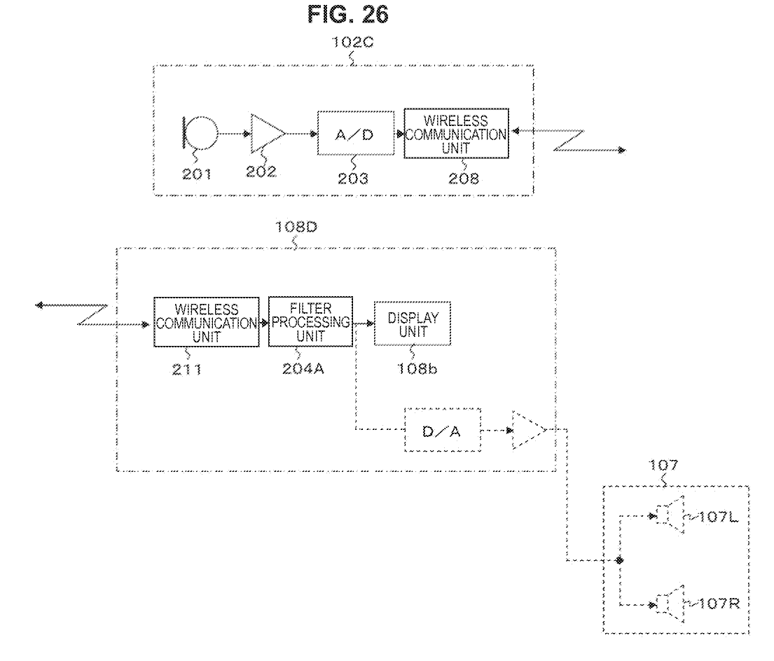

FIG. 26 illustrates another configuration example of the acoustic processing unit 102C and the display device 108D. In FIG. 24, structural elements that correspond to the structural elements of FIGS. 14 and 20 are denoted with the same reference numerals and repeated explanation of these structural elements is omitted. Similar to the acoustic processing unit 102C of FIG. 20, the acoustic processing unit 102C has a microphone 201, an amplifier 202, an A/D converter 203, and a wireless communication unit 208.

The display device 108D has a wireless communication unit 211, a filter processing unit 204A, and a display unit 108b. The wireless communication unit 211 performs communication between the acoustic processing unit 102C and the wireless communication unit 211. That is, the wireless communication unit 211 receives the acoustic signal (vibration signal) that is transmitted from the acoustic processing unit 102C. The wireless communication unit 211 performs communication for wireless connection processing between the acoustic processing unit 102C and the wireless communication unit 211.

The filter processing unit 204A has a correction filter to perform filtering of a filter characteristic including an inverse characteristic of an entire acoustic characteristic or a partial acoustic characteristic of the chest piece 101 and the microphone 201. By the filtering, deterioration of a frequency characteristic in each portion such as the chest piece 101 can be compensated for. The display unit 108b displays a waveform and/or a frequency spectrum, based on the corrected acoustic

Next, operations of the acoustic processing unit 102C and the display device 108D illustrated in FIG. 26 will be described. The acoustic signal that is converted by the A/D converter 203 of the acoustic processing unit 102C from an analog signal to a digital signal is supplied to the wireless communication unit 208. The acoustic signal (vibration signal) is wirelessly transmitted from the wireless communication unit 208 to the display device 108D. In the wireless communication unit 211 of the display device 108D, the acoustic signal (vibration signal) that is transmitted from the acoustic processing unit 102C is received. The acoustic signal is supplied to the filter processing unit 204A.

In the filter processing unit 204A, filtering of a filter characteristic including an inverse characteristic of an entire acoustic characteristic or a partial acoustic characteristic of the chest piece 101 and the microphone 201 is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is supplied to the display unit 108b. The waveform and/or the frequency spectrum is displayed on the display unit 108b, based on the corrected acoustic signal.

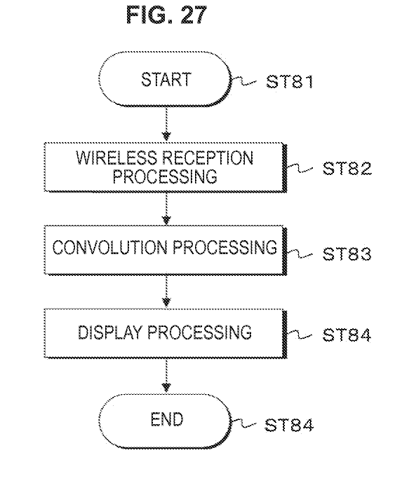

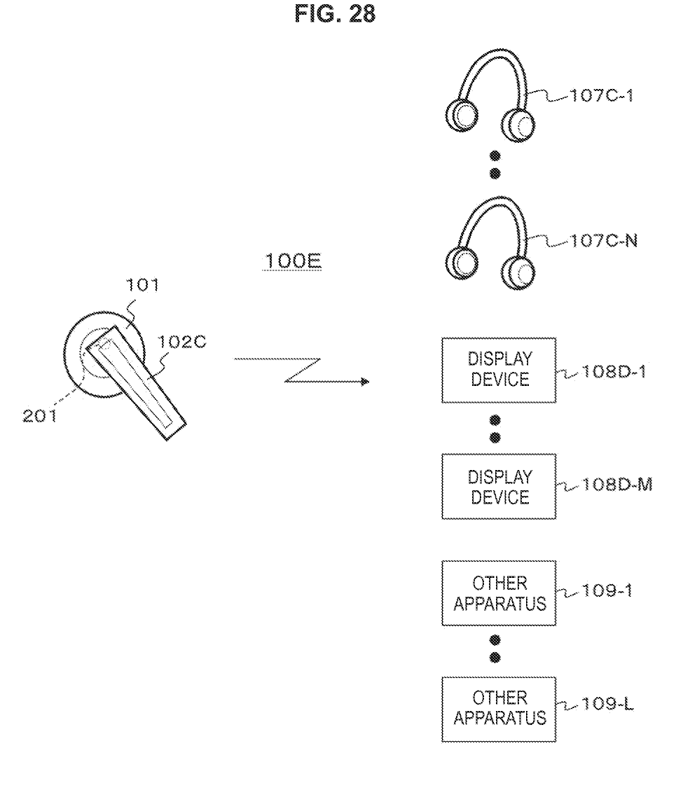

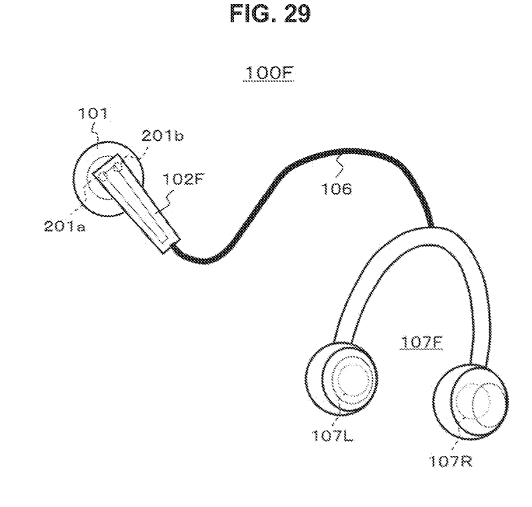

A flowchart of FIG. 27 illustrates a processing sequence of the display device 108D illustrated in FIG. 26. The display device 108D starts processing in step ST81 and proceeds to processing of step ST82. In step ST82, the display device 108D receives the acoustic signal (vibration signal) transmitted from the acoustic processing unit 102C, by the wireless communication unit 211.

Next, in step ST83, the display device 108D convolutes a correction filter coefficient (impulse response) on the received acoustic signal and performs filtering of a correction filter characteristic. In step ST84, the display device 108D displays the waveform and/or the frequency spectrum, based on the corrected acoustic signal. Then, in step ST85, the display device 108D ends the processing.

In the processing sequence illustrated in the flowchart of FIG. 27, the display device 108D executes convolution processing on a time axis in step ST83. However, the convolution processing may be executed on the frequency axis. When a tap length increases, the convolution processing is executed on the frequency axis, so that an operation amount can be decreased and an operation load can be alleviated.

As described above, in the vibration detecting apparatus 100D illustrated in FIG. 23, filtering of a filter characteristic including an inverse characteristic of an entire acoustic characteristic or a partial acoustic characteristic of the chest piece 101 and the microphone 201 is executed by the acoustic processing unit 102C. For this reason, deterioration of a frequency characteristic in each portion such as the chest piece 101 can be compensated for and a user can observe a waveform or a frequency spectrum of a biological vibration such as a cardiac sound or a pulmonary sound with a superior characteristic, using the display device 108D.

As described above, in the vibration detecting apparatus 100D that displays the waveform and/or frequency spectrum of the corrected acoustic signal on the display device 108D, as shown by a broken line in FIG. 23, the headphone 107 is connected to the display device 108D, so that the user can hear a sound obtained from the corrected acoustic signal, that is, a biological vibration sound such as a cardiac sound or a pulmonary sound with a superior characteristic. In this case, for the headphone 107, the display device 108D constitutes a repeater.

In the case of the display device 108D illustrated in FIG. 24, as shown by a broken line, the acoustic signal that is received by the wireless communication unit 211 is converted by the D/A converter from a digital signal to an analog signal, is amplified by the amplifier, and is supplied to the speakers 107L and 107R constituting the headphone 107. In the case of the display device 108D illustrated in FIG. 26, as shown by a broken line, the corrected acoustic signal that is output from the filter processing unit 204A is converted by the D/A converter from a digital signal to an analog signal, is amplified by the amplifier, and is supplied to the speakers 107L and 107R constituting the headphone 107.

### 6. Sixth Embodiment

### Configuration Example of Vibration Detecting Apparatus

FIG. 28 illustrates a configuration example of a vibration detecting apparatus 100E according to a sixth embodiment. In FIG. 28, structural elements that correspond to the structural elements of FIGS. 16 and 23 are denoted with the same reference numerals and repeated explanation of these structural elements is omitted. The vibration detecting apparatus 100E includes a chest piece 101, an acoustic processing unit 102C, N headphones 107C-1 to 107C-N, M display devices 108D-1 to 108D-M, and L other apparatuses 109-1 to 109-L.

The vibration detecting apparatus 100E has a configuration in which a plurality of external apparatuses (headphones, display devices, and other apparatuses) are wirelessly connected to the acoustic processing unit 102C to which the chest piece 101 is connected. In this case, other apparatuses are electronic apparatuses using an acoustic signal (vibration signal) transmitted from the acoustic processing unit 102C. For example, other apparatuses include a sound output apparatus, an electronic medical chart generating apparatus, and a diagnosis supporting apparatus other than the headphones.

Although detailed explanation is omitted, the acoustic processing unit 102C has the same configuration as the acoustic processing unit 102C illustrated in FIG. 17 or 20 described above. In this case, a point-to-multipoint wireless communication system is adopted as a wireless communication system.

Each of the headphones 107C-1 to 107C-N has the same configuration as the headphone 107C illustrated in FIG. 17 or 20 described above. Each of the display devices 108D-1 to 108D-M has the same configuration as the display device 108D illustrated in FIG. 24 or 26 described above. Each of other apparatuses 109-1 to 109-L includes a wireless communication unit that receives an acoustic signal (vibration signal) transmitted from the acoustic processing unit 102C, like the headphone 107C or the display device 108D.

As described above, in the vibration detecting apparatus 100E illustrated in FIG. 28, an acoustic signal (vibration signal) that is wirelessly transmitted from one acoustic processing unit 102C can be used by a plurality of external apparatuses at the same time.

### 7. Seventh Embodiment

### Configuration Example of Vibration Detecting Apparatus

FIG. 29 illustrates a configuration example of a vibration detecting apparatus 100F according to a seventh embodiment. In FIG. 29, structural elements that correspond to the structural elements of FIG. 9 are denoted with the same reference numerals and repeated explanation of these structural elements is omitted. The vibration detecting apparatus 100F includes a chest piece 101, an acoustic processing unit 102F, a connection line 106, and a headphone 107F.

The vibration detecting apparatus 100F has a configuration in which the headphone 107F is connected to the acoustic processing unit 102F to which the chest piece 101 is connected, through the connection line 106. The acoustic processing unit 102F has a plurality of microphones, in this embodiment, two microphones 201a and 201b. Each of the microphones is mounted on a different position of the chest piece 101. Correction of an acoustic characteristic (a frequency characteristic and a phase characteristic) is performed with respect to an acoustic signal (vibration signal) obtained by each microphone and the corrected acoustic signal is supplied to the headphone 107F that a user wears, through the connection line 106.

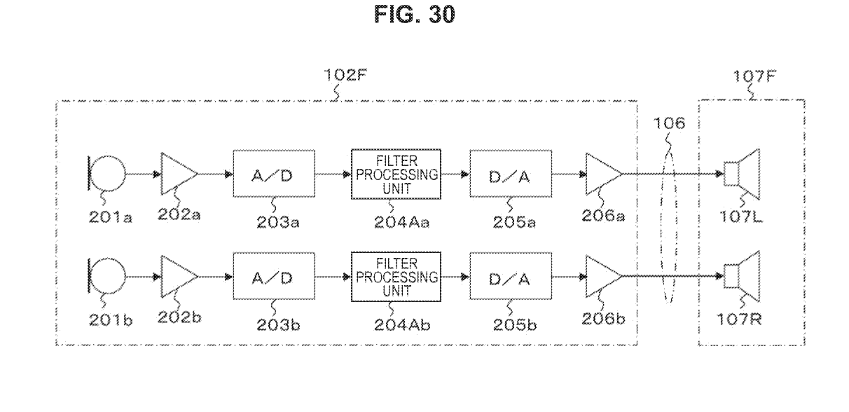

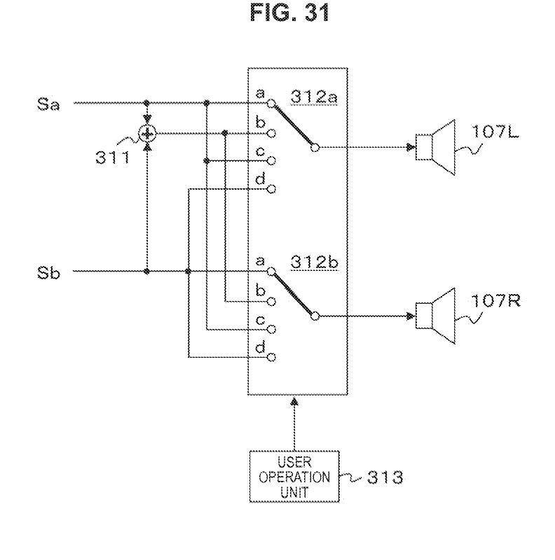

FIG. 30 illustrates a configuration example of the acoustic processing unit 102F and the headphone 107F. In FIG. 30, structural elements that correspond to the structural elements of FIG. 10 are denoted with the same reference numerals and repeated explanation of these structural elements is omitted. The acoustic processing unit 102E has a microphone 201a, an amplifier 202a, an A/D converter 203a, a filter processing unit 204Aa, a D/A converter 205a, and an amplifier 206a as a system of an a channel. The acoustic processing unit 102F has a microphone 201b, an amplifier 202b, an A/D converter 203b, a filter processing unit 204Ab, a D/A converter 205b, and an amplifier 206b as a system of a b channel.

Each of the microphones 201a and 201b is mounted on a different position of the chest piece 101. Each of the microphones 201a and 201b converts a sound (vibration) collected by the chest piece 101 into an acoustic signal (vibration signal) to be an electric signal. The microphones 201a and 201b constitute a biological vibration detecting unit together with the chest piece 101.

The amplifiers 202a and 202b amplify the acoustic signals that are acquired by the microphones 201a and 201b, respectively. The A/D converters 203a and 203b convert the acoustic signals output from the amplifiers 202a and 202b from analog signals to digital signals. The filter processing units 204Aa and 204Ab perform filtering to correct an acoustic characteristic (a frequency characteristic and a phase characteristic) with respect to the acoustic signals output from the A/D converters 203a and 203b, respectively.

In this case, correction filter coefficients (impulse responses) of correction filters of the filter processing units 204Aa and 204Ab may be set differently or equally. When the correction filter coefficient is set differently for each channel, the correction filter coefficient according to the system of each channel can be set for each channel. Therefore, an acoustic signal (vibration signal) of each channel can be accurately corrected. Meanwhile, when the correction filter coefficient is set commonly to each channel, only one kind of correction filter coefficient may be stored. Therefore, a memory amount can be decreased.

The D/A converters 205a and 205b convert the acoustic signals output from the filter processing units 204Aa and 204Ab from digital signals to analog signals. The amplifiers 206a and 206h amplify the acoustic signals of the a channel and the b channel output from the D/A converters 205a and 205b and transmit the acoustic signals to the headphone 107F through the connection line 106.

The headphone 107F has a left speaker 107L and a right speaker 107R. The headphone 107F receives the acoustic signals of the a channel and the b channel through the connection line 106 and supplies the acoustic signals to the speakers 107L and 107R, respectively.

Next, operations of the acoustic processing unit 102F and the headphone 107F illustrated in FIG. 30 will be described. In the microphones 201a and 201b of the acoustic processing unit 102F, sounds (vibrations) that are collected by the chest piece 101 (refer to FIG. 29) are converted into electric signals and the acoustic signals (vibration signals) of the a channel and the b channel are obtained. The acoustic signals of the individual channels are amplified by the amplifiers 202a and 202b, are converted the A/D converters 203a and 203b from analog signals to digital signals, and are supplied to the filter processing units 204Aa and 204Ab.

In the filter processing units 204Aa and 204Ab, filtering of a filter characteristic including an inverse characteristic of an entire acoustic characteristic or a partial acoustic characteristic of the chest piece 101, the microphones 201a and 201b, and the speakers 107L and 107R is performed. By the filtering, for each channel, deterioration of a frequency characteristic in each portion such as the chest piece 101 is compensated for. In the filter processing units 204Aa and 204Ab, filtering to remove noise and filtering to convert an acoustic characteristic into a desired acoustic characteristic may be performed.

The corrected acoustic signals of the a channel and the b channel that are output from the filter processing units 204Aa and 204Ab are converted by the D/A converters 205a and 205b from digital signals to analog signals and are amplified by the amplifiers 206a and 206b. The amplified acoustic signals (vibration signals) of the a channel and the b channel are supplied to the left and right speakers 107L and 107R constituting the headphone 107F, through the connection line 106. Sounds (vibrations) that are obtained from the corrected acoustic signals of the a channel and the b channel are output from the speakers 107L and 107R, respectively.

As described above, in the vibration detecting apparatus 100F illustrated in FIG. 29, filtering of a filter characteristic including an inverse characteristic of an entire acoustic characteristic or a partial acoustic characteristic of the chest piece 101, the microphones 201a and 201b, and the speakers 107L and 107R is executed by the acoustic processing unit 102F. For this reason, deterioration of a frequency characteristic in each portion such as the chest piece 101 can be compensated for and a user can hear a biological vibration sound such as a cardiac sound or a pulmonary sound detected by each of the systems of the a channel and the b channel with a superior characteristic, using the headphone 107F.

In the headphone 107F illustrated in FIG. 30, a biological vibration sound of the a channel is output from the left speaker 107L and a biological vibration sound of the b channel is output from the right speaker 107R. As output states of the speakers 107L and 107R, any one of the following output states (1) to (4) may be used or a plurality of output states thereof may be selectively switched.

(1) One of the speakers 107L and 107R outputs the biological vibration sound of the a channel and the other outputs the biological vibration sound of the b channel.
(2) Both the speakers 107L and 107R output a synthetic biological vibration sound of the a channel and the b channel.
(3) Both the speakers 107L and 107R output the biological vibration sound of the a channel.
(4) Both the speakers 107L and 107R output the biological vibration sound of the b channel.

FIG. 31 illustrates a configuration example of the headphone 107F in which the output states (1) to (4) can be selectively switched. The headphone 107F has a synthesizing unit 311, switches 312a and 312b, a user operation unit 313, and speakers 107L and 107R.

The synthesizing unit 311 synthesizes the acoustic signals (vibration signals) of the a channel and the b channel that are supplied from the acoustic processing unit 102F. The switch 312a selectively outputs the acoustic signal (vibration signal) to be supplied to the speaker 107L. The switch 312b selectively outputs the acoustic signal (vibration signal) to be supplied to the speaker 107R. The user operation unit 313 switches the switches 312a and 312b in an interlocked manner, based on the user operation.

An acoustic signal (vibration signal) Sa of the a channel that is supplied from the acoustic processing unit 102F is supplied to the synthesizing unit 311, a-side and c-side fixed terminals of the switch 312a, and a c-side fixed terminal of the switch 312b. An acoustic signal (vibration signal) Sb of the b channel that is supplied from the acoustic processing unit 102F is supplied to the synthesizing unit 311, a-side and d-side fixed terminals of the switch 312b, and a d-side fixed terminal of the switch 312a. An acoustic signal (vibration signal) that is output from the synthesizing unit 311 is supplied to a b-side fixed terminal of the switch 312a and a b-side fixed terminal of the switch 312b.

When each of the switches 312a and 312b is switched into the a-side, the acoustic signal Sa of the a channel is supplied to the speaker 107L through the switch 312a and the acoustic signal Sb of the b channel is supplied to the speaker 107R through the switch 312b. For this reason, the biological vibration sound of the a channel is output from the speaker 107L and the biological vibration sound of the b channel is output from the speaker 107R.

When each of the switches 312a and 312b is switched into the b-side, the synthetic acoustic signal of the a channel and the b channel is supplied to the speakers 107L and 107R through the switches 312a and 312b and the acoustic signal Sb of the b channel is supplied to the speaker 107R through the switch 312b. For this reason, the synthetic biological vibration sound of the a channel and the b channel is output from both the speakers 107L and 107R.

When each of the switches 312a and 312b is switched into the c-side, the acoustic signal Sa of the a channel is supplied to the speakers 107L and 107R through the switches 312a and 312b. For this reason, the biological vibration sound of the a channel is output from both the speakers 107L and 107R.

When each of the switches 312a and 312b is switched into the d-side, the acoustic signal Sb of the b channel is supplied to the speakers 107L and 107R through the switches 312a and 312b. For this reason, the biological vibration sound of the b channel is output from both the speakers 107L and 107R.

As described above, the headphone 107F is configured as illustrated in FIG. 31 so that a user can switch the output states of the speakers 107L and 107R, according to necessity. For this reason, the user can appropriately switch the biological vibration sound of each channel and the synthetic biological vibration sound of each channel and compare the biological vibration sounds by hearing the biological vibration sounds. As a result, the user can diagnose illness with high precision.

In the vibration detecting apparatus 100F illustrated in FIG. 29, the acoustic processing unit 102F and the headphone 107F are connected by the connection line 106. However, the acoustic processing unit 102F and the headphone 107F may be wirelessly connected.

### 8. Eighth Embodiment

### Configuration Example of Vibration Detecting Apparatus

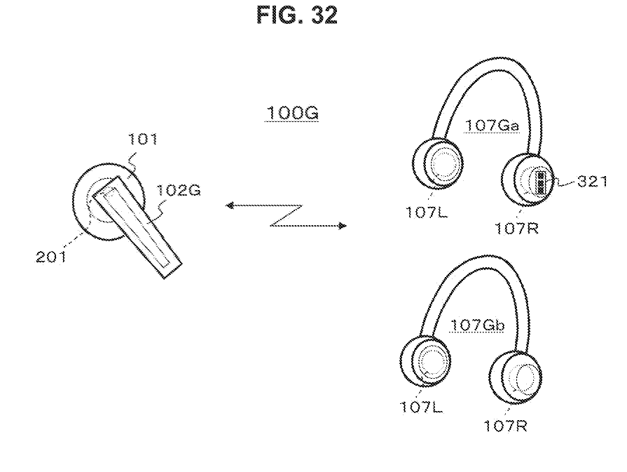

FIG. 32 illustrates a configuration example of a vibration detecting apparatus 100G according to an eighth embodiment. In FIG. 32, structural elements that correspond to the structural elements of FIG. 16 are denoted with the same reference numerals and repeated explanation of these structural elements is omitted. The vibration detecting apparatus 100G includes a chest piece 101, an acoustic processing unit 102G, a headphone 107Ga, and a headphone 107Gb.

The vibration detecting apparatus 100G has a configuration in which the headphones 107Ga and 107Gb are wirelessly connected to the acoustic processing unit 102G to which the chest piece 101 is connected. The acoustic processing unit 102G has a microphone 201 and performs correction of an acoustic characteristic (a frequency characteristic and a phase characteristic) with respect to a sound collected by the chest piece 101 and wirelessly transmits the corrected acoustic signal to the headphones 107Ga and 107Gb that a user wears.

For example, near field communication such as Bluetooth is used as wireless communication. The headphones 107Ga and 107Gb execute authentication processing for wireless connection between the acoustic processing unit 102G and the headphones 107Ga and 107Gb, according to a user operation or automatically, if necessary, and enter a wireless connection state. For example, in the case of the Bluetooth, pairing is performed or released based on the user operation.

The headphone 107Ga has a user operation unit 321. A user of the headphone 107Ga operates the user operation unit 321 and can control transmission of an acoustic signal (vibration signal) from the acoustic processing unit 102G to the headphone 107Gb.

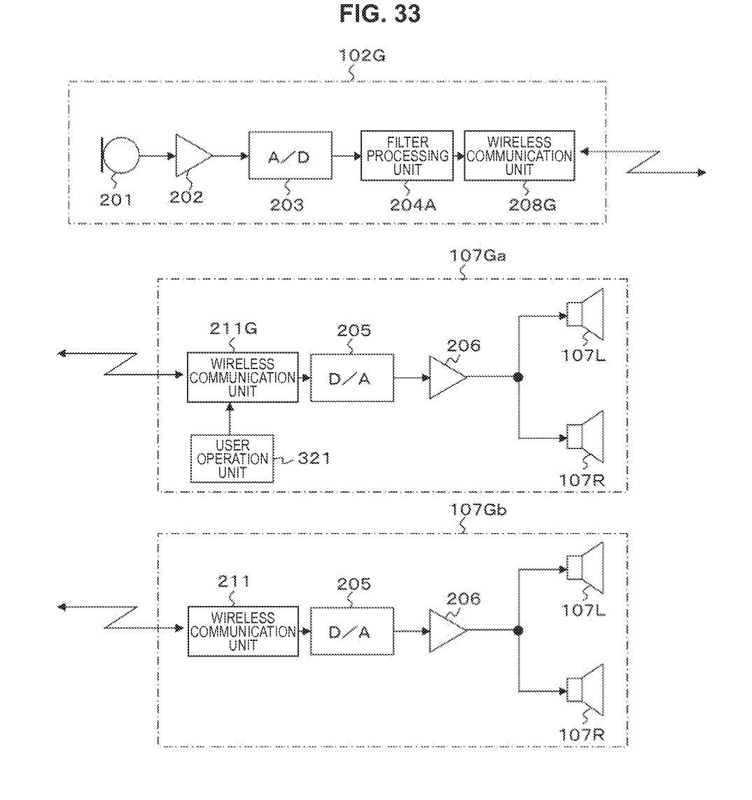

FIG. 33 illustrates a configuration example of the acoustic processing unit 102G and the headphones 107Ga and 107Gb. In FIG. 33, structural elements that correspond to the structural elements of FIG. 17 are denoted with the same reference numerals and repeated explanation of these structural elements is omitted. The acoustic processing unit 102G has a microphone 201, an amplifier 202, an A/D converter 203, a filter processing unit 204A, and a wireless communication unit 208G.

The filter processing unit 204A has a correction filter to perform filtering of a filter characteristic including an inverse characteristic of an entire acoustic characteristic or a partial acoustic characteristic of the chest piece 101, the microphone 201, and speakers 107L and 107R. By the filtering, deterioration of a frequency characteristic in each portion such as the chest piece 101 can be compensated for.

The wireless communication unit 208G performs communication between the headphones 107Ga and 107Gb and the wireless communication unit 208G. That is, the wireless communication unit 208G transmits the corrected acoustic signal (vibration signal) output from the filter processing unit 204A to the headphones 107Ga and 107Gb. The wireless communication 208G performs communication for wireless connection processing between the headphone 107C and the wireless communication unit 208G in this case, a point-to-multipoint wireless communication system is adopted as a wireless communication system.

The wireless communication unit 208G receives the user operation signal in the headphone 107Ga and selectively transmits the corrected acoustic signal (vibration signal) to the headphone 107Gb, based on the user operation signal. That is, the wireless communication unit 208G transmits the acoustic signal to the headphone 107Gb, when a user operation signal shows “transmission” and does not transmit the acoustic signal to the headphone 107Gb, when the user operation signal shows “non-transmission”.

The headphone 107Ga has a wireless communication unit 211G, a D/A converter 205, an amplifier 206, speakers 107L and 107R, and a user operation unit 321. The wireless communication unit 211G performs communication between the acoustic processing unit 102G and the wireless communication unit 211G. That is, the wireless communication unit 211G receives the corrected acoustic signal (vibration signal) that is transmitted from the acoustic processing unit 102G. The wireless communication unit 211G performs communication for wireless connection processing between the acoustic processing unit 102C and the wireless communication unit 211G.

The wireless communication unit 211G transmits a user operation signal generated from the user operation unit 321 to correspond to the user operation, to the acoustic processing unit 102G. The user operation signal is a signal to control transmission of the acoustic signal (vibration signal) from the wireless communication unit 208G to the headphone 107Gb and shows “transmission” or “non-transmission”.

The D/A converter 205 converts the acoustic signal received by the wireless communication unit 211G from a digital signal to an analog signal. The amplifier 206 amplifies the acoustic signal output from the D/A converter 205 and supplies the acoustic signal to the speakers 107L and 107R.

The headphone 107Gb has a wireless communication unit 211, a D/A converter 205, an amplifier 206, and speakers 207L and 207R. Although detailed explanation is omitted, the headphone 107Gb has the same configuration as the headphone 107C illustrated in FIG. 17 described above.

Next, operations of the acoustic processing unit 102G and the headphones 107Ga and 107Gb illustrated in FIG. 33 will be described. In the microphone 201 of the acoustic processing unit 102G, a sound (vibration) that is collected by the chest piece 101 (refer to FIG. 32) is converted into an acoustic signal (vibration signal) to be an electric signal. The acoustic signal is amplified by the amplifier 202, is converted by the A/D converter 203 from an analog signal to a digital signal, and is supplied to the filter processing unit 204A.

In the filter processing unit 204A, filtering of a filter characteristic including an inverse characteristic of an entire acoustic characteristic or a partial acoustic characteristic of the chest piece 101, the microphone 201, and the speakers 107L and 107R is performed. By the filtering, deterioration of a frequency characteristic in each portion such as the chest piece 101 is compensated for. In the filter processing unit 204A, filtering to remove noise and filtering to convert an acoustic characteristic into a desired acoustic characteristic may be performed.

The corrected acoustic signal output from the filter processing unit 204A is supplied to the wireless communication unit 208G. The corrected acoustic signal is wirelessly transmitted from the wireless communication unit 208G to the headphones 107Ga and 107Gb.

In the wireless communication unit 211G of the headphone 107Ga, the corrected acoustic signal (vibration signal) that is transmitted from the acoustic processing unit 102G is received. The acoustic signal is converted by the D/A converter 205 from a digital signal to an analog signal, is amplified by the amplifier 206, and is supplied to the left and right speakers 107L and 107R. A sound (vibration) that is obtained from the corrected acoustic signal is output from these speakers 107L and 107R.

In the wireless communication unit 211 of headphone 107Gb, the corrected acoustic signal (vibration signal) that is transmitted from the acoustic processing unit 102G is received. The acoustic signal is converted by the D/A converter 205 from a digital signal to an analog signal, is amplified by the amplifier 206, and is supplied to the left and right speakers 107L and 107R. A sound (vibration; that is obtained from the corrected acoustic signal is output from these speakers 107L and 107R.

In a state in which the acoustic signal is transmitted from the acoustic processing unit 102G to the headphone 107Gb, in the headphone 107Ga, the user operation signal showing “non-transmission” can be transmitted to the acoustic processing unit 102G, based on a user operation with respect to the user operation unit 321. In this case, in the acoustic processing unit 102G, the user operation signal is received and the acoustic signal is not transmitted to the headphone 107Gb.

In a state in which the acoustic signal is not transmitted from the acoustic processing unit 102G to the headphone 107Gb, in the headphone 107Ga, the user operation signal showing “transmission” can be transmitted to the acoustic processing unit 102G, based on the user operation with respect to the user operation unit 321. In this case, in the acoustic processing unit 102G, the user operation signal is received and the acoustic signal is transmitted to the headphone 107Gb.

As described above, in the vibration detecting apparatus 100G illustrated in FIG. 32, filtering of a filter characteristic including an inverse characteristic of an entire acoustic characteristic or a partial acoustic characteristic of the chest piece 101, the microphone 201, and the speakers 107L and 107R is executed by the acoustic processing unit 102G. For this reason, deterioration of a frequency characteristic in each portion such as the chest piece 101 can be compensated for and a user can hear a biological vibration sound such as a cardiac sound or a pulmonary sound with a superior characteristic, using the headphones 107Ga and 107Gb.

In the vibration detecting apparatus 100G illustrated in FIG. 32, a user of the headphone 107Ga operates the user operation unit 321 and can control transmission of an acoustic signal (vibration signal) from the acoustic processing unit 102G to the headphone 107Gb. For this reason, a person (for example, a doctor and a teacher) who wears the headphone 107Ga and hears a biological vibration sound can arbitrarily set whether or not to allow a person (for example, a patient and a student) who wears the headphone 107Gb to hear the biological vibration sound.

### 9. Ninth Embodiment

### Configuration Example of Vibration Detecting Apparatus

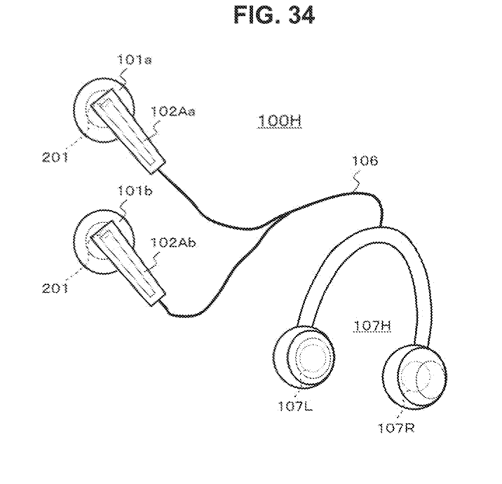

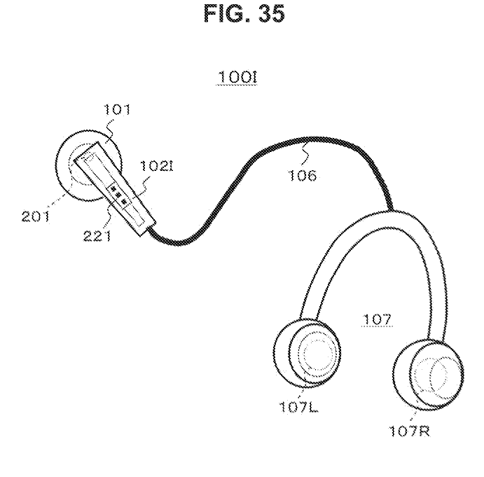

FIG. 34 illustrates a configuration example of a vibration detecting apparatus 100H according to a ninth embodiment. In FIG. 34, structural elements that correspond to the structural elements of FIGS. 9 and 29 are denoted with the same reference numerals and repeated explanation of these structural elements is omitted. The vibration detecting apparatus 100H includes chest pieces 101a and 101b, acoustic processing units 102Aa and 102Ab, a connection line 106, and a headphone 107H.

The vibration detecting apparatus 100H has a configuration in which the headphone 107H is connected to the acoustic processing unit 102Aa to which the chest piece 101a is connected and the acoustic processing unit 102Ab to which the chest piece 101b is connected, through the connection line 106. Each of the acoustic processing units 102Aa and 102A b has the same configuration as the acoustic processing unit 102A (refer to FIG. 10) in the vibration detecting apparatus 100A illustrated in FIG. 9. The headphone 107H has the same configuration as the headphone 107F (refer to FIGS. 30 and 31) in the vibration detecting apparatus 100F illustrated in FIG. 29,

An acoustic characteristic corrected acoustic signal (vibration signal) that is output from the acoustic processing unit 102Aa is supplied to the headphone 107H through the connection line 106. In addition, an acoustic characteristic corrected acoustic signal (vibration signal) that is output from the acoustic processing unit 102Ab is supplied to the headphone 107H through the connection line 106.

When the headphone 107H has the same configuration as the headphone 107F illustrated in FIG. 30, output states of the speakers 107L and 107R constituting the headphone 107H are as follows. That is, a biological vibration sound that is obtained from the corrected acoustic signal (vibration signal) supplied from the acoustic processing unit 102Aa is output from the left speaker 107L. Meanwhile, a biological vibration sound that is obtained from the corrected acoustic signal (vibration signal) supplied from the acoustic processing unit 102Ab is output from the right speaker 107R.

Meanwhile, when the headphone 107H has the same configuration as the headphone 107F illustrated in FIG. 31, the user can switch the output states of the speakers 107L and 107R according to necessity. That is, the user can switch the output states of the speakers 107L and 107R into a state in which only one biological vibration sound is output or a state in which a synthetic biological vibration sound is output.

As described above, in the vibration detecting apparatus 100H illustrated in FIG. 34, the acoustic processing unit 102Aa connected to the chest piece 101a and the acoustic processing unit 102Ab connected to the chest piece 101b are included and the biological vibration can be independently detected in each of the acoustic processing unit 102Aa and the acoustic processing unit 102Ab. Filtering of a filter characteristic including an inverse characteristic of an entire acoustic characteristic or a partial acoustic characteristic of the chest pieces 101a and 101b, the microphones 201a and 201b, and the speakers 107L and 107R is executed by the acoustic processing units 102Aa and 102Ab. For this reason, deterioration of a frequency characteristic in each portion such as the chest pieces 101a and 101b can be compensated for and a user can hear biological vibration sounds such as cardiac sounds or pulmonary sounds detected in two places with a superior characteristic, using the headphone 107H.

In the above description, each of the acoustic processing units 102Aa and 102Ab has the same configuration as the acoustic processing unit 102A of the vibration detecting apparatus 100A illustrated in FIG. 9 and includes the filter processing unit 204A. Although detailed explanation is omitted, each of the acoustic processing units 102Aa and 102Ab may not include the filter processing unit 204A and the filter processing unit 204A may be arranged in the headphone 107H.

In the vibration detecting apparatus 100H illustrated in FIG. 34, the acoustic processing units 102Aa and 102Ab and the headphone 107H are connected by the connection line 106. However, the acoustic processing units 102Aa and 102Ab and the headphone 107H may be wirelessly connected.

### 10. Tenth Embodiment

### Configuration Example of Vibration Detecting Apparatus

FIG. 35 illustrates a configuration example of a vibration detecting apparatus 100I according to a tenth embodiment. In FIG. 35, structural elements that correspond to the structural elements of FIG. 9 are denoted with the same reference numerals and repeated explanation of these structural elements is omitted. The vibration detecting apparatus 100I includes a chest piece 101, an acoustic processing unit 102I, a connection line 106, and a headphone 107.

The vibration detecting apparatus 100I has a configuration in which the headphone 107 is connected to the acoustic processing unit 102I to which the chest piece 101 is connected, through the connection line 106. The acoustic processing 102I has one microphone 201 that is mounted on the chest piece 101. Correction of an acoustic characteristic (a frequency characteristic and a phase characteristic) is performed with respect to an acoustic signal (vibration signal) obtained by the microphone 201 and the corrected acoustic signal is supplied to the headphone 107 that a user wears, through the connection line 106.

The acoustic processing unit 102I has a user operation unit 221. A user operates the user operation unit 221 and can switch a filter characteristic of a correction filter. In the switching of the filter characteristic, a range of a correction amount or a frequency to be flattened changes. In this case, the filter characteristic can be set to an optimal filter characteristic, according to kinds of biological objects (people, dogs, horses, and elephants) or biological vibrations (an organ sound such as a cardiac sound and a pulmonary sound and a respiratory sound such as a snoring sound). Even when the same person is targeted, the filter characteristic can be changed according to a physical type such as a thickness of fat.

The filter characteristic can be changed according to the change of the pressing pressure of a diaphragm of the chest piece 101. The filter characteristic can be changed according to an environmental situation such as noise. The switching of the filter characteristic of the correction filter can be performed based on the user operation. In addition, the switching of the filer characteristic of the correction filter can be performed automatically based on outputs of various sensors, according to setting of the user, if necessary.

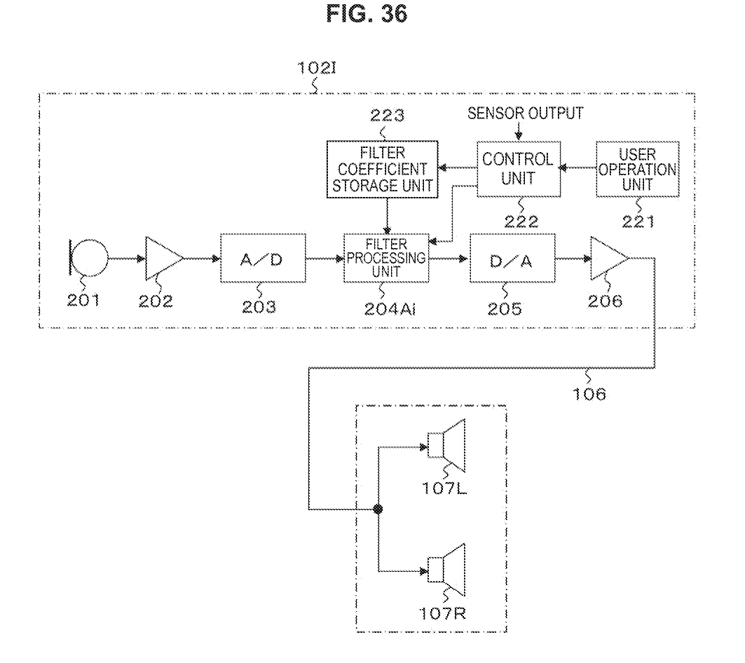

FIG. 36 illustrates a configuration example of the acoustic processing unit 102I and the headphone 107. In FIG. 36, structural elements that correspond to the structural elements of FIG. 10 are denoted with the same reference numerals and repeated explanation of these structural elements is omitted. The acoustic processing unit 102I has a microphone 201, an amplifier 202, an A/D converter 203, a filter processing unit 204Ai, a D/A converter 205, an amplifier 206, a user operation unit 221, a control unit 222, and a filter coefficient storage unit 223.

The filter processing unit 204Ai performs filtering to correct an acoustic characteristic (a frequency characteristic and a phase characteristic) with respect to the acoustic signal (vibration signal) output from the A/D converter 203. The filter processing unit 204Ai has a correction filter to perform filtering of a filter characteristic including an inverse characteristic of an entire acoustic characteristic or a partial acoustic characteristic of the chest piece 101, the microphone 201, and the speakers 107L and 107R. By the filtering, deterioration of a frequency characteristic in each portion such as the chest piece 101 can be compensated for.

For example, the filter processing unit 204Ai has a correction filter that performs filtering to remove noise and a correction filer that performs filtering to convert an acoustic characteristic into a desired acoustic characteristic. Similar to the filter processing unit 204A of the acoustic processing unit 102A of FIG. 10, the filtering is performed by executing an impulse response convolution operation with respect to the acoustic signal.

The D/A converter 205 converts an acoustic signal output from the filter processing unit 204Ai from a digital signal to an analog signal. The amplifier 206 amplifies the acoustic signal output from the D/A converter 205 and supplies the acoustic signal to the left speaker 107L and the right speaker 107R constituting the headphone 107, through the connection line 106.

The user operation unit 221 is used to switch the filter characteristic of the correction filter. For example, the user operates the user operation unit 221 and inputs information of kinds of biological objects (people, dogs, horses, and elephants) or biological vibrations (an organ sound such as a cardiac sound and a pulmonary sound and a respiratory sound such as a snoring sound). Also, the user inputs environmental information such as noise. Also, the user operates the user operation unit 221 and selects a filter characteristic of a specific kind from a plurality of kinds of filter characteristics.

The filter coefficient storage unit 223 stores filter coefficients (impulse responses) that correspond to a plurality of kinds of correction filters. The control unit 222 extracts an appropriate kind of filter coefficient (impulse response) from the filter coefficient storage unit 223, based on the information from the user operation unit 221 and/or the various sensor outputs, and sets the filter coefficient to the filter processing unit 204Ai. The sensor outputs include an output of a noise sensor and an output of a pressure sensor detecting the pressing pressure of the diaphragm.

Next, operations of the acoustic processing unit 102I and the headphone 107 illustrated in FIG. 36 will be described. The information of the kinds of the biological objects or the biological vibrations input based on the user operation with respect to the user operation unit 221 or the selection information of the filter coefficient is supplied to the control unit 222. The various sensor outputs are supplied to the control unit 222. In the control unit 222, the filter characteristic of the correction filter to be used is determined based on the information described above. Linder the control from the control unit 222, the filter coefficient (impulse response) of the specific kind corresponding to the filter characteristic of the determined correction filter is extracted from the filter coefficient storage unit 233 and is set to the filter processing unit 204Ai.

In the microphone 201 of the acoustic processing unit 102I, a sound (vibration) that is collected by the chest piece 101 (refer to FIG. 35) is converted into an acoustic signal (vibration signal) to be an electric signal. The acoustic signal is amplified by the amplifier 202, is converted by the A/D converter 203 from an analog signal to a digital signal, and is supplied to the filter processing unit 204Ai.

In the filter processing unit 204Ai, filtering of the correction filter based on the filter coefficient (impulse response) set as described above is performed. By the filtering, deterioration of a frequency characteristic in each portion such as the chest piece 101 is compensated for. By the filtering, a desired acoustic characteristic according to the kinds of the biological objects or the biological vibrations is obtained. By the filtering, removing of the environmental noise is also performed.

The corrected acoustic signal output from the filter processing unit 204Ai is converted by the D/A converter 205 from a digital signal to an analog signal, is amplified by the amplifier 206, and is supplied to the left and right speakers 107L and 107R constituting the headphone 107, through the connection line 106. A sound (vibration) that is obtained from the corrected acoustic signal is output from these speakers 107L and 107R.

As described above, in the vibration detecting apparatus 100I illustrated in FIG. 35, filtering of a filter characteristic including an inverse characteristic of an entire acoustic characteristic or a partial acoustic characteristic of the chest piece 101, the microphone 201, and the speakers 107L and 107R is executed by the acoustic processing unit 102I. For this reason, deterioration of a frequency characteristic in each portion such as the chest piece 101 can be compensated for and a user can hear a biological vibration sound such as a cardiac sound or a pulmonary sound with a superior characteristic, using the headphone 107.

in the vibration detecting apparatus 100I illustrated in FIG. 35, the filter characteristic of the correction filter can be switched according to the kinds of the biological objects or the biological vibrations. Therefore, the biological vibrations can be securely detected with a desired acoustic characteristic according to the kinds of the biological objects or the biological vibrations.

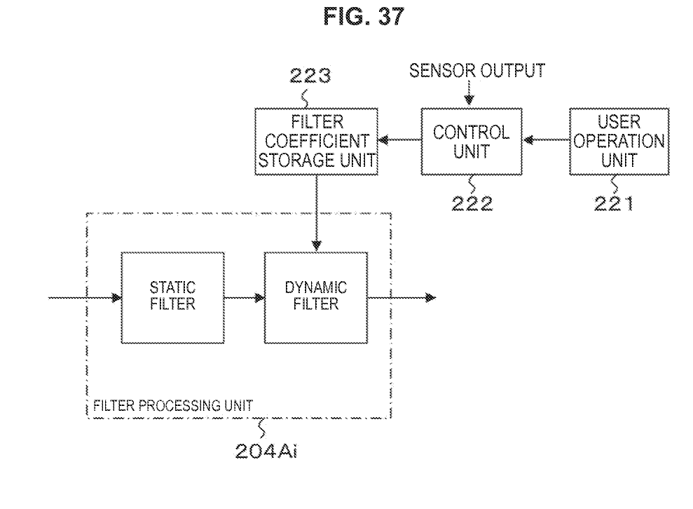

When the correction filters included in the filter processing unit 204Ai include a static filter having a fixed filter characteristic and a dynamic filter having a variable filter characteristic, as illustrated in FIG. 37, only the filter characteristic of the dynamic filter may be switched.

In the acoustic processing unit 102I of FIG. 36, the filter coefficient is selectively extracted from the filter coefficient storage unit 223 and is set to the filter processing unit 204Ai, so that the filter characteristic of the correction filter is switched. However, an appropriate filter coefficient may be downloaded from an external apparatus (server) connected to a network and may be set to the filter processing unit 204Ai, so that the filter characteristic of the correction filter may be switched.

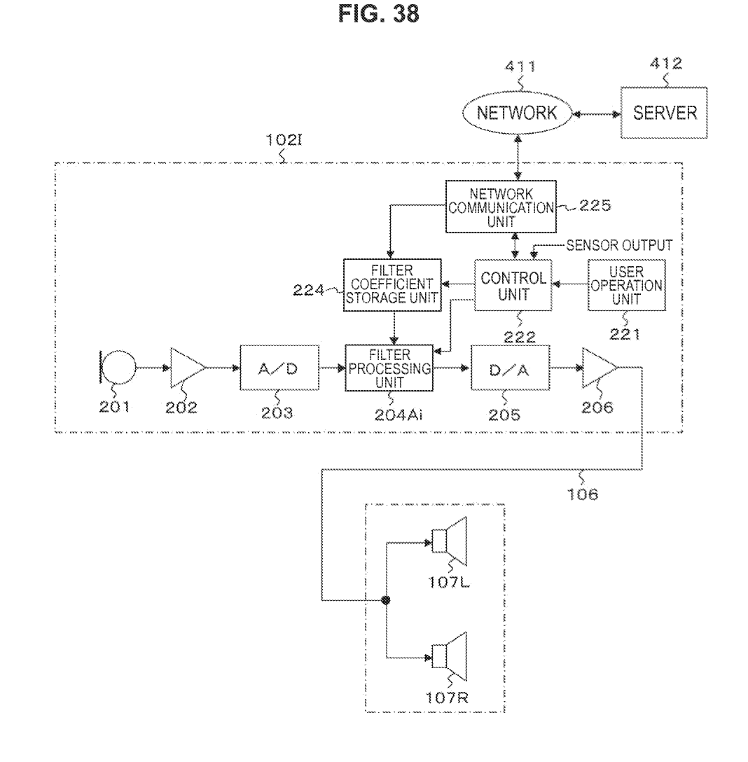

FIG. 38 illustrates a configuration example of the acoustic processing unit 102I and the headphone 107. In FIG. 38, structural elements that correspond to the structural elements of FIG. 36 are denoted with the same reference numerals and repeated explanation of these structural elements is omitted. The acoustic processing unit 102I has a microphone 201, an amplifier 202, an AD converter 203, a filter processing unit 204Ai, a D/A converter 205, an amplifier 206, a user operation unit 221, a control unit 222, a filter coefficient storage unit 224, and a network communication unit 225.

The filter coefficient storage unit 224 stores a filter coefficient (impulse response) that is downloaded from the server 412 on the network 411 by the network communication unit 225. The control unit 222 controls the network communication unit 225, based on information from the user operation unit 221 and/or various sensor outputs, and causes the network communication unit 225 to download a filter coefficient (impulse response) of an appropriate kind. The control unit 222 sets the filter coefficient (impulse response), which has been downloaded and has been stored in the filter coefficient storage unit 224, to the filter processing unit 204Ai.

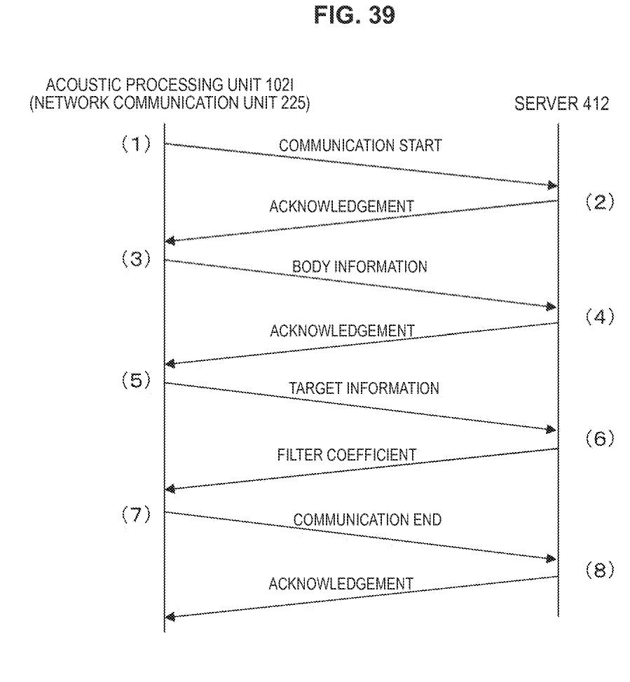

The network communication unit 225 downloads the filter coefficient (impulse response) from the server 412 on the network 411, under the control from the control unit 222. A sequence diagram of FIG. 39 illustrates an example of a communication sequence between the network communication unit 225 of the acoustic processing unit 102I and the server 412 functioning as an external apparatus.

(1) The network communication unit 225 transmits a communication start command to the server 412. (2) With respect to a communication start request, the server 412 transmits an acknowledgement to the network communication unit 225. (3) Next, the network communication unit 225 transmits body information to the server 412. The body information is information used to determine a filter characteristic of a correction filter portion (static correction filter portion) correcting a characteristic of the earpiece, the microphone, or the speaker, in which the filter characteristic does not change. The body information includes model number information of the earpiece. (4) The server 412 transmits an acknowledgement to the network communication unit 225 to correspond to the information transmission.

(5) Next, the network communication unit 225 transmits target information to the server 412. The target information is information used to determine a filter characteristic of a correction filter portion (dynamic correction filter portion) correcting the characteristic according to a use state or an environmental situation. The target information includes the information from the user operation unit 221 or the sensor outputs. (6) With respect to the transmission of the information, the server 412 transmits a filter coefficient determined based on the body information and the target information to the network communication unit 225.

(7) Next, the network communication unit 225 transmits a communication end command to the server 412. (8) With respect to the transmission of the communication end command, the server 412 transmits an acknowledgement to the network communication unit 225.

Next, operations of the acoustic processing unit 102I and the headphone 107 illustrated in FIG. 38 will be described. The information of the kinds of the biological objects or the biological vibrations input based on the user operation with respect to the user operation unit 221 is supplied to the control unit 222. The various sensor outputs are supplied to the control unit 222.

The control unit 222 controls the network communication unit 225 and causes the network communication unit 225 to download the filter coefficient (impulse response) corresponding to the information (target information) and the body information from the server 412 on the network 411. The downloaded filter coefficient is stored in the filter coefficient storage unit 224. Under the control from the control unit 222, the filter coefficient is set to the filter processing unit 204Ai.

In the microphone 201 of the acoustic processing unit 102I, a sound ration) that is collected by the chest piece 101 (refer to FIG. 35) is converted into an acoustic signal (vibration signal) to be an electric signal. The acoustic signal is amplified by the amplifier 202, is converted by the A/D converter 203 from an analog signal to a digital signal, and is supplied to the filter processing unit 204Ai.

In the filter processing unit 204Ai, filtering of the correction filter based on the filter coefficient (impulse response) set as described above is performed. By the filtering, deterioration of a frequency characteristic in each portion such as the chest piece 101 is compensated for. By the filtering, a desired acoustic characteristic according to the kinds of the biological objects or the biological vibrations is obtained. By the filtering, removing of the environmental noise is also performed.

The corrected acoustic signal output from the filter processing unit 204Ai is converted by the D/A converter 205 from a digital signal to an analog signal, is amplified by the amplifier 206, and is supplied to the left and right speakers 107L and 107R constituting the headphone 107, through the connection line 106. A sound (vibration) that is obtained from the corrected acoustic signal is output from these speakers 107L and 107R.

In the sequence diagram of FIG. 39, the body information and the target information are transmitted from the network communication unit 225 to the server 412. However, a necessary filter coefficient may be specified by the control unit 222 based on the body information and the target information and information showing the specified filter coefficient may be transmitted from the network communication unit 225 of the acoustic processing unit 102I to the server 412.

In the vibration detecting apparatus 100I illustrated in FIG. 35, the acoustic processing unit 102I and the headphone 107 are connected by the connection line 106. However, the acoustic processing unit 102I and the headphone 107 may be wirelessly connected.

In the vibration detecting apparatus 100I illustrated in FIG. 35, the acoustic processing unit 102I and the headphone 107 are connected by the connection line 106. However, instead of the headphone 107, a display device and the acoustic processing unit 102I may be connected (refer to FIG. 11).

### 11. Eleventh Embodiment

### Configuration Example of Vibration Detecting Apparatus

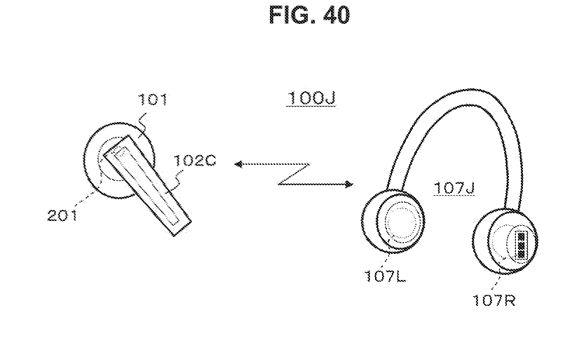

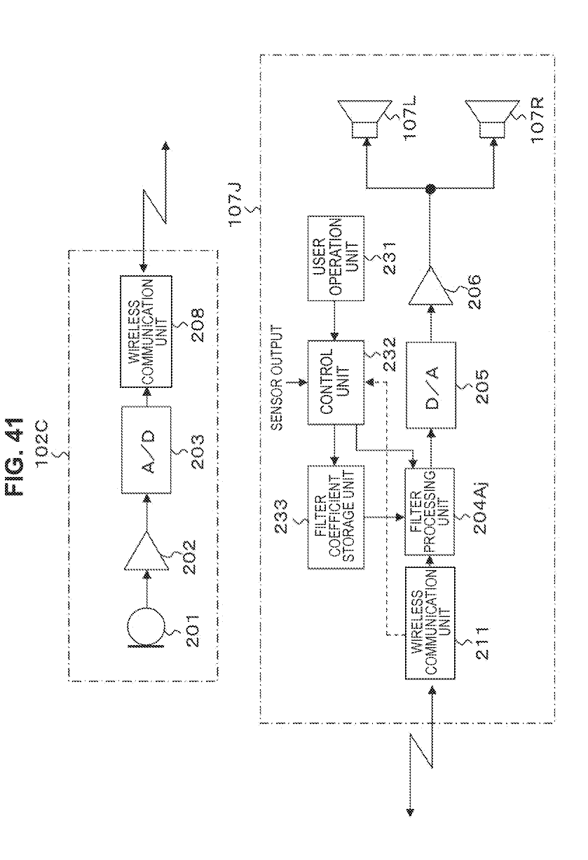

FIG. 40 illustrates a configuration example of a vibration detecting apparatus 100J according to an eleventh embodiment. In FIG. 40, structural elements that correspond to the structural elements of FIG. 16 are denoted with the same reference numerals and repeated explanation of these structural elements is omitted. The vibration detecting apparatus 100J includes a chest piece 101, an acoustic processing unit 102C, and a headphone 107J.

The vibration detecting apparatus 100J has a configuration in which the headphone 107J is wirelessly connected to the acoustic processing unit 102C to which the chest piece 101 is connected. The acoustic processing unit 102C has a microphone 201 and performs correction of an acoustic characteristic (a frequency characteristic and a phase characteristic) with respect to a sound collected by the chest piece 101 and wirelessly transmits the corrected acoustic signal to the headphone 107J that a user wears.

The headphone 107J has a user operation unit 231. A user operates the user operation unit 231 and can switch a filter characteristic of a correction filter. In the switching of the filter characteristic, a range of a correction amount or a frequency to be flattened changes. In this case, the filter characteristic can be set to an optimal filter characteristic, according to kinds of biological objects (people, dogs, horses, and elephants) or biological vibrations (an organ sound such as a cardiac sound and a pulmonary sound and a respiratory sound such as a snoring sound). Even when the same person is targeted, the filter characteristic can be changed according to a physical type such as a thickness of fat.

The filter characteristic can be changed according to the change of the pressing pressure of a diaphragm of the chest piece 101. The filter characteristic can be changed according to an environmental situation such as noise. The switching of the filter characteristic of the correction filter can be performed based on the user operation. In addition, the switching of the filer characteristic of the correction filter can be performed automatically based on outputs of various sensors, according to setting of the user, if necessary.

FIG. 41 illustrates a configuration example of the acoustic processing unit 102C and the headphone 107J. In FIG. 41, structural elements that correspond to the structural elements of FIG. 20 are denoted with the same reference numerals and repeated explanation of these structural elements is omitted. Similar to the acoustic processing unit 102C of FIG. 20, the acoustic processing unit 102C has a microphone 201, an amplifier 202, an A/D converter 203, and a wireless communication unit 208.

The headphone 107J has a wireless communication unit 211, a filter processing unit 204Aj, a D/A converter 205, an amplifier 206, speakers 107L and 107R, a user operation unit 231, a control unit 232, and a filter coefficient storage unit 233. The wireless communication unit 211 performs communication between the acoustic processing unit 102C and the wireless communication unit 211. That is, the wireless communication unit 211 receives an acoustic signal (vibration signal) that is transmitted from the acoustic processing unit 102C.

The wireless communication unit 211 performs communication for wireless connection processing between the acoustic processing unit 102C and the wireless communication unit 211, at the time of wireless connection with the acoustic processing unit 102C. At this time, the wireless communication unit 211 acquires information used to determine a filter characteristic of a correction filter portion correcting a characteristic of the side of the acoustic processing unit 102C such as the earpiece 101 and the microphone 201, for example, model number information of the earpiece, from the acoustic processing unit 102C, and transmits the information to the control unit 232. When the wireless communication unit 211 receives an acoustic signal (vibration signal) from the acoustic processing unit 102C, the wireless communication unit 211 may also receive a sensor output showing the pressing pressure of a diaphragm and transmit the sensor output to the control unit 232.

The filter processing unit 204Aj performs filtering to correct an acoustic characteristic (a frequency characteristic and a phase characteristic) with respect to an acoustic signal (vibration signal) received by the wireless communication unit 211. The filter processing unit 204Aj has a correction filter to perform filtering of a filter characteristic including an inverse characteristic of an entire acoustic characteristic or a partial acoustic characteristic of the chest piece 101, the microphone 201, and the speakers 107L and 107R. By the filtering, deterioration of a frequency characteristic in each portion such as the chest piece 101 can be compensated for.

For example, the filter processing unit 204Ai has a correction filter that performs filtering to remove noise and a correction filer that performs filtering to convert an acoustic characteristic into a desired acoustic characteristic. Similar to the filter processing unit 204A of the acoustic processing unit 102C of FIG. 20, the filtering is performed by executing an impulse response convolution operation with respect to an acoustic signal.

The D/A converter 205 converts an acoustic signal output from the filter processing unit 204Aj from a digital signal to an analog signal. The amplifier 206 amplifies the acoustic signal output from the D/A converter 205 and supplies the acoustic signal to the left speaker 107L and the right speaker 107R constituting the headphone 107, through the connection line 106.

The user operation unit 231 is used to switch the filter characteristic of the correction filter. For example, a user operates the user operation unit 231 and inputs information of kinds of biological objects (people, dogs, horses, and elephants) or biological vibrations (an organ sound such as a cardiac sound or a pulmonary sound and a respiratory sound such as a snoring sound). Also, the user inputs environmental information such as noise. Also, the user operates the user operation unit 231 and selects a filter characteristic of a specific kind from a plurality of kinds of filter characteristics.

The filter coefficient storage unit 233 stores filter coefficients (impulse responses) that correspond to a plurality of kinds of correction filters. The control unit 232 extracts an appropriate kind of filter coefficient (impulse response) from the filter coefficient storage unit 233, based on the information from the user operation unit 231, the various sensor outputs, and the information of the side of the acoustic processing unit 102C received by the wireless communication unit 211, and sets the filter coefficient to the filter processing unit 204Aj. The sensor outputs that are supplied directly to the control unit 232 include an output of a noise sensor.

Next, operations of the acoustic processing unit 102C and the headphone 107J illustrated in FIG. 41 will be described. The information of the kinds of the biological objects or the biological vibrations input based on the user operation with respect to the user operation unit 231 or the selection information of the filter coefficient is supplied to the control unit 232. The various sensor outputs are supplied to the control unit 232. The information of the side of the acoustic processing unit 102C that is received by the wireless communication unit 211 is also supplied to the control unit 232.

In the control unit 232, the filter characteristic of the correction filter to be used is determined based on the information described above. Under the control from the control unit 232, the filter coefficient (impulse response) of the specific kind corresponding to the filter characteristic of the determined correction filter is extracted from the filter coefficient storage unit 233 and is set to the filter processing unit 204Aj.

In the microphone 201 of the acoustic processing unit 102C, a sound (vibration) that is collected by the chest piece 101 (refer to FIG. 40) is converted into an acoustic signal (vibration signal) to be an electric signal. The acoustic signal is amplified by the amplifier 202 and is converted by the A/D converter 203 from an analog signal to a digital signal. The acoustic signal is transmitted from the wireless communication unit 208 to the headphone 107L

In the wireless communication unit 211 of the headphone 107J, the acoustic signal (vibration signal) that is transmitted from the acoustic processing unit 102C is received. The acoustic signal is supplied to the filter processing unit 204Aj. In the filter processing unit 204Aj, filtering of the correction filter based on the filter coefficient (impulse response) set as described above is performed. By the filtering, deterioration of a frequency characteristic in each portion such as the chest piece 101 is compensated for. By the filtering, a desired acoustic characteristic according to the kinds of the biological objects or the biological vibrations is obtained. By the filtering, removing of the environmental noise is also performed.

The corrected acoustic signal output from the filter processing unit 204Aj is converted by the D/A converter 205 from a digital signal to an analog signal, is amplified by the amplifier 206, and is supplied to the left and right speakers 107L, and 107R. A sound (vibration) that is obtained from the corrected acoustic signal is output from these speakers 107L and 107R.

As described above, in the vibration detecting apparatus 100J illustrated in FIG. 40, filtering of a filter characteristic including an inverse characteristic of an entire acoustic characteristic or a partial acoustic characteristic of the chest piece 101, the microphone 201, and the speakers 107L and 107R is executed by the headphone 107J. For this reason, deterioration of a frequency characteristic in each portion such as the chest piece 101 can be compensated for and a user can hear a biological vibration sound such as a cardiac sound or a pulmonary sound with a superior characteristic, using the headphone 107J.

In the vibration detecting apparatus 100J illustrated in FIG. 40, the filter characteristic of the correction filter can be switched according to the kinds of the biological objects or the biological vibrations. Therefore, the biological vibration can be securely detected with a desired acoustic characteristic according to the kinds of the biological objects or the biological vibrations.

When tire correction filters included in the filter processing unit 204Aj include a static filter having a fixed filter characteristic and a dynamic filter having a variable filter characteristic, only the filter characteristic of the dynamic filter may be switched (refer to FIG. 37).

In the headphone 107J of FIG. 41, the filter coefficient is selectively extracted from the filter coefficient storage unit 233 and is set to the filter processing unit 204Aj, so that the filter characteristic of the correction filter is switched. However, an appropriate filter coefficient may be downloaded from an external apparatus (server) connected to a network and may be set to the filter processing unit 204Aj, so that the filter characteristic of the correction filter may be switched.

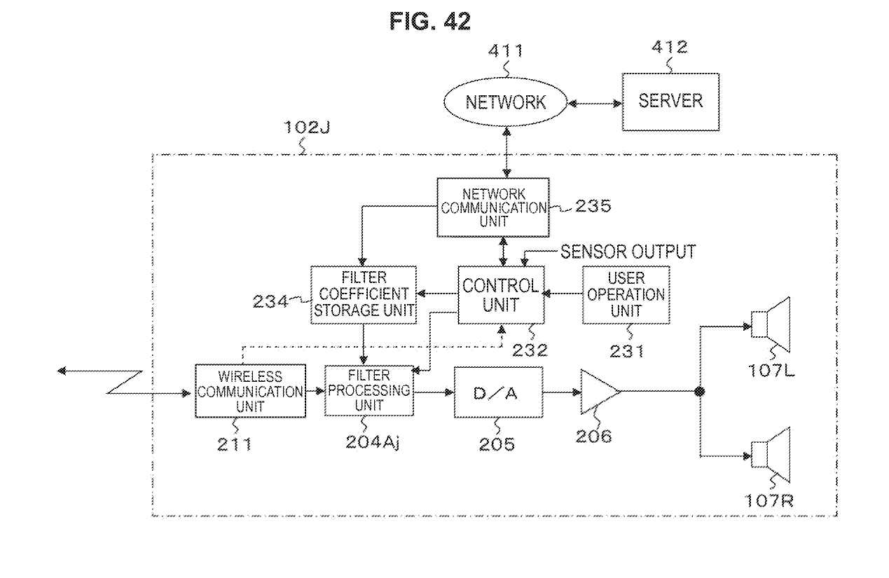

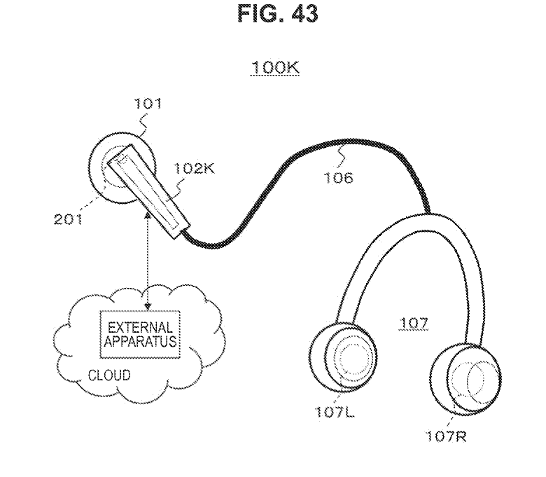

FIG. 42 illustrates a configuration example of the headphone 107J. In FIG. 42, structural elements that correspond to the structural elements of FIGS. 36 and 41 are denoted with the same reference numerals and repeated explanation of these structural elements is omitted. The headphone 107J has a wireless communication unit 211, a filter processing unit 204Aj, a D/A converter 205, an amplifier 206, speakers 107L and 107R, a user operation unit 231, a control unit 232, a filter coefficient storage unit 234, and a network communication unit 235.

The network communication unit 235 downloads the filter coefficient (impulse response) from the server 412 on the network 411, under the control from the control unit 232. The filter coefficient storage unit 234 stores the downloaded filter coefficient. The control unit 232 controls the network communication unit 235, based on information from the user operation unit 231, various sensor outputs, and information of the side of the acoustic processing unit 102C received by the wireless communication unit 211, and causes the network communication unit 235 to download a filter coefficient of an appropriate kind. The control unit 232 sets the filter coefficient, which has been downloaded and has been stored in the filter coefficient storage unit 234, to the filter processing unit 204Aj.

Next, an operation of the headphone 107J illustrated in FIG. 42 will be described. The information of the kinds of the biological objects or the biological vibrations input based on the user operation with respect to the user operation unit 231 is supplied to the control unit 232. The various sensor outputs are supplied to the control unit 232. The information of the side of the acoustic processing unit 102C that is received by the wireless communication unit 211 is supplied to the control unit 232.

The control unit 232 controls the network communication unit 235 and causes the network communication unit 235 to download the filter coefficient (impulse response) corresponding to the information from the server 412 on the network 411. The downloaded filter coefficient is stored in the filter coefficient storage unit 234. Under the control from the control unit 232, the filter coefficient is set to the filter processing unit 204Aj.

In the wireless communication unit 211, the acoustic signal (vibration signal) that is transmitted from the acoustic processing unit 102C is received. The acoustic signal is supplied to the filter processing unit 204Aj. In the filter processing unit 204Aj, filtering of the correction filter based on the filter coefficient (impulse response) set as described above is performed. By the filtering, deterioration of a frequency characteristic in each portion such as the chest piece 101 is compensated for. By the filtering, a desired acoustic characteristic according to the kinds of the biological objects or the biological vibrations is obtained. By the filtering, removing of the environmental noise is also performed.

The corrected acoustic signal output from the filter processing unit 204Aj is converted by the D/A converter 205 from a digital signal to an analog signal, is amplified by the amplifier 206, and is supplied to the left and right speakers 107L and 107R. A sound (vibration) that is obtained from the corrected acoustic signal is output from these speakers 107L and 107R.

In the vibration detecting apparatus 100J illustrated in FIG. 40, the acoustic processing unit 102C and the headphone 107J are wirelessly connected. However, instead of the headphone 107J, a display device and the acoustic processing unit 102C may be connected (refer to FIG. 23).

### 12. Twelfth Embodiment

### Configuration Example of Vibration Detecting Apparatus

FIG. 43 illustrates a configuration example of a vibration detecting apparatus 100K according to a twelfth embodiment. In FIG. 43, structural elements that correspond to the structural elements of FIG. 10 are denoted with the same reference numerals and repeated explanation of these structural elements is omitted. The vibration detecting apparatus 100K includes a chest piece 101, an acoustic processing unit 102K, a connection line 106, and a headphone 107.

The vibration detecting apparatus 100K has a configuration in which the headphone 107 is connected to the acoustic processing unit 102K to which the chest piece 101 is connected, through the connection line 106. The acoustic processing unit 102K has one microphone 201 that is mounted on the chest piece 101. Correction of an acoustic characteristic (a frequency characteristic and a phase characteristic) is performed with respect to an acoustic signal (vibration signal) obtained by the microphone 201 and the corrected acoustic signal is supplied to the headphone 107 that a user wears, through the connection line 106.

The filtering of the correction filter is performed at the side of the acoustic processing unit 102K. The acoustic processing unit 102K does not directly perform the filtering of the correction filter and uses a filtering function in an external apparatus on a cloud. In this case, the acoustic processing unit 102K performs communication for the filtering of the correction filter between the external apparatus on the cloud, that is, the external apparatus connected to a network and the acoustic processing unit 102K.

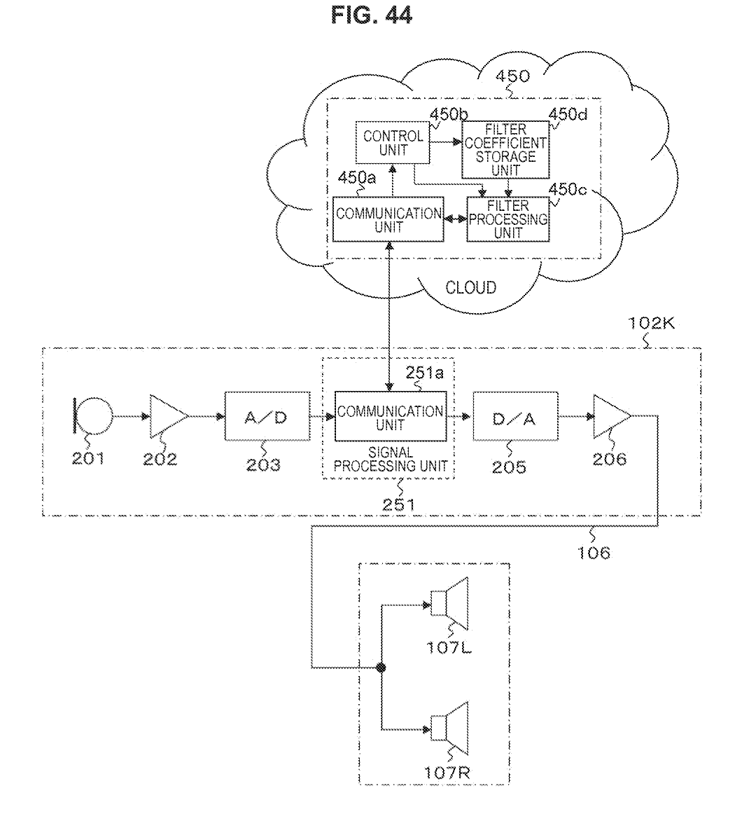

FIG. 44 illustrates a configuration example of the vibration detecting apparatus 100K and the headphone 107. In FIG. 44, structural elements that correspond to the structural elements of FIG. 10 are denoted with the same reference numerals and repeated explanation of these structural elements is omitted. The acoustic processing unit 102K has a microphone 201, an amplifier 202, an A/D converter 203, a signal processing unit 251, a D/A converter 205, and an amplifier 206.

The microphone 201 is mounted on the chest piece 101 (refer to FIG. 43). The microphone 201 converts a sound (vibration) collected by the chest piece 101 into an acoustic signal (vibration signal) to be an electric signal. The microphone 201 constitutes a biological vibration detecting unit together with the chest piece 101. The amplifier 202 amplifies the acoustic signal that is acquired by the microphone 201. The A/D converter 203 converts the acoustic signal output from the amplifier 202 from an analog signal to a digital signal.

The signal processing unit 251 has a communication unit 251a. The communication unit 251a transmits the acoustic signal (vibration signal) output from the A/D converter 203 to an external apparatus 450 on the cloud. The communication unit 251a receives a corrected acoustic signal (vibration signal) that is obtained by the external apparatus 450 on the cloud.

The external apparatus 450 has a communication unit 450a, a control unit 450b, a filter processing unit 450c, and a filter coefficient storage unit 450d. The communication unit 450 performs communication with the communication unit 251a of the signal processing unit 251 of the acoustic processing unit 102K. The communication unit 450a receives a non-corrected acoustic signal (vibration signal) transmitted from the communication unit 251a and transmits the acoustic signal to the filter processing unit 450c.

The communication unit 450a receives body information or target information transmitted from the communication unit 251a and transmits the body information or the target information to the control unit 450b. The body information is information used to determine a filter characteristic of a correction filter portion (static correction filter portion) correcting a characteristic of the earpiece, the microphone, or the speaker, in which the filter characteristic does not change. The body information includes model number information of the earpiece. The target information is information used to determine a filter characteristic of a connection filter portion (dynamic correction filter portion) correcting the characteristic according to a use state or an environmental situation. The target information includes user operation information showing the use state or the environmental situation or sensor outputs.

The filter coefficient storage unit 450d stores filter coefficients (impulse responses) that correspond to a plurality of kinds of correction filters. The control unit 450b extracts an appropriate kind of filter coefficient (impulse response) from the filter coefficient storage unit 450d, based on the body information or the target information received by the communication unit 450a, and sets the filter coefficient to the filter processing unit 450c.

The filter processing unit 450c performs filtering of the correction filter based on the filter coefficient (impulse response) set as described above, with respect to the acoustic signal (vibration signal) received by the communication unit 450a. By the filtering, deterioration of a frequency characteristic in each portion such as the chest piece 101 is compensated for, a desired acoustic characteristic according to the kinds of the biological objects or the biological vibrations is obtained, and removing of the environmental noise is also performed. The communication unit 450c transmits the acoustic signal (vibration signal) corrected by the filter processing unit 450c to the communication unit 251a.

The D/A converter 205 converts the corrected acoustic signal (vibration signal) received by the communication unit 251a from a digital signal to an analog signal. The amplifier 206 amplifies the acoustic signal output from the D/A converter 205 and supplies the acoustic signal to the left speaker 107L and the right speaker 107R constituting the headphone 107, through the connection line 106.

Next, operations of the acoustic processing unit 102K and the headphone 107 illustrated in FIG. 44 will be described. In the microphone 201 of the acoustic processing unit 102K, a sound (vibration) that is collected by the chest piece 101 (refer to FIG. 43) is converted into an acoustic signal (vibration signal) to be an electric signal. The acoustic signal is amplified by the amplifier 202, is converted by the A/D converter 203 from an analog signal to a digital signal, and is supplied to the signal processing unit 251. In the signal processing unit 251, the acoustic signal that is supplied from the A/D converter 203 is transmitted to the external apparatus 450 on the cloud by the communication unit 251a. At this time, the body information or the target information is transmitted together with the acoustic signal,

In the external apparatus 450, filtering of the correction filter based on the filter coefficient (impulse response) selected based on the body information or the target information is performed by the filter processing unit 450c. The acoustic signal is transmitted from the external apparatus 450 to the communication unit 251a. The corrected acoustic signal that is received by the communication unit 251a is converted by the D/A converter 205 from a digital signal to an analog signal, is amplified by the amplifier 206, and is supplied to the speakers 107L and 107R constituting the headphone 107, through the connection line 106. A sound (vibration) that is obtained from the corrected acoustic signal is output from these speakers 107L and 107R.

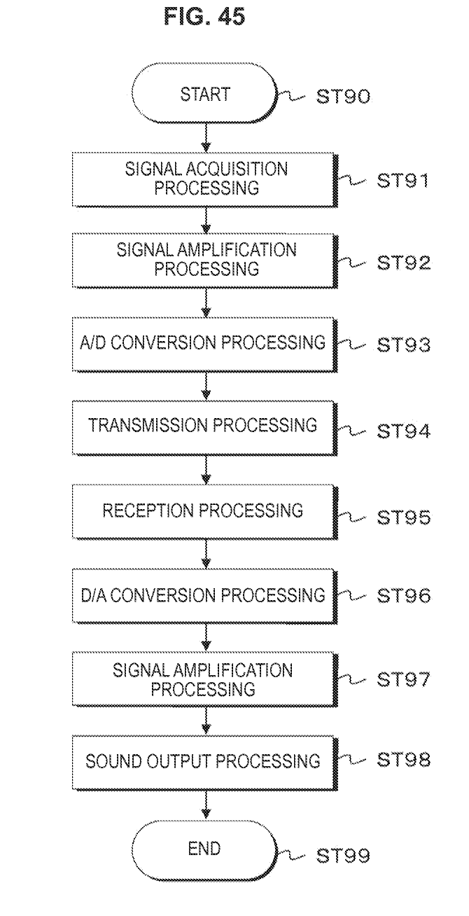

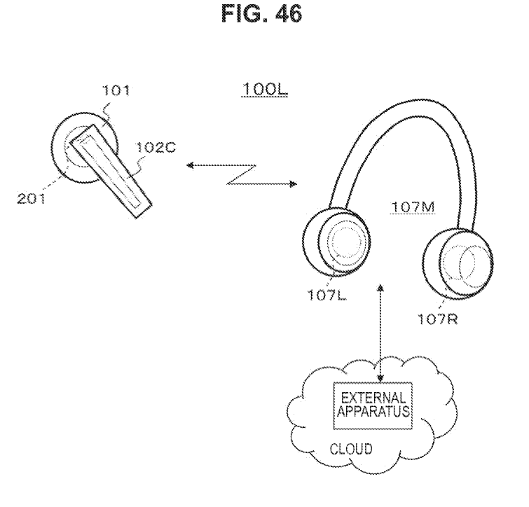

A flowchart of FIG. 45 illustrates a processing sequence of the acoustic processing unit 102K and the headphone 107 illustrated in FIG. 44. The acoustic processing unit 102K and the headphone 107 start processing in step ST90 and proceed to processing of step ST91. In step ST91, the acoustic processing unit 102K acquires an acoustic signal (vibration signal) corresponding to a sound (vibration) collected by the chest piece 101, by the microphone 201.

Next, in step ST92, the acoustic processing unit 102K amplifies the acoustic signal acquired by the microphone 201. In step ST93, the acoustic processing unit 102K converts the amplified acoustic signal from an analog signal to a digital signal. In step ST94, the acoustic processing unit 102K transmits the acoustic signal converted into the digital signal to the external apparatus 450 on the cloud, by the communication unit 215a of the signal processing unit 251.

Next, in step ST95, the acoustic processing unit 102K receives the corrected acoustic signal that is transmitted from the external apparatus 450 on the cloud, by the communication unit 215a of the signal processing unit 251. In step ST96, the acoustic processing unit 102K converts the corrected acoustic signal from a digital signal to an analog signal. In step ST97, the acoustic processing unit 102K amplifies the acoustic signal and supplies the acoustic signal to the headphone 107.

Next, in step ST98, the headphone 107 outputs a sound (vibration) obtained from the corrected acoustic signal from the speakers 107L and 107R. Then, in step ST99, the acoustic processing unit 102K and the headphone 107 end the processing.

As described above, in the vibration detecting apparatus 100K illustrated in FIG. 43, filtering of a filter characteristic including an inverse characteristic of an entire acoustic characteristic or a partial acoustic characteristic of the chest piece 101, the microphone 201, and the speakers 107L and 107R is executed by the external apparatus 450 on the cloud. For this reason, deterioration of a frequency characteristic in each portion such as the chest piece 101 can be compensated for and a user can hear a biological vibration sound such as a cardiac sound or a pulmonary sound with a superior characteristic, using the headphone 107.

In the vibration detecting apparatus 100K illustrated in FIG. 43, the acoustic processing unit 102K does not directly perform the filtering and uses a filtering function in the external apparatus 450 on the cloud. For this reason, a biological vibration signal having a superior characteristic can be obtained without providing a correction filter having a heavy processing load in the acoustic processing unit 102K.

In the vibration detecting apparatus 100K illustrated in FIG. 43, the acoustic processing unit 102K and the headphone 107 are connected by the connection line 106. However, the acoustic processing unit 102K and the headphone 107 may be wirelessly connected.

In the vibration detecting apparatus 100K illustrated in FIG. 43, the acoustic processing unit 102K and the headphone 107 are connected by the connection line 106. However, instead of the headphone 107, a display device and the acoustic processing unit 102K may be connected (refer to FIG. 11).

### 13. Thirteenth Embodiment

### Configuration Example of Vibration Detecting Apparatus

FIG. 46 illustrates a configuration example of a vibration detecting apparatus 100L according to a thirteenth embodiment. In FIG. 46, structural elements that correspond to the structural elements of FIG. 16 are denoted with the same reference numerals and repeated explanation of these structural elements is omitted. The vibration detecting apparatus 100L includes a chest piece 101, an acoustic processing unit 102C, and a headphone 107M.

The vibration detecting apparatus 100L has a configuration in which the headphone 107M is wirelessly connected to the acoustic processing unit 102C to which the chest piece 101 is connected. The acoustic processing unit 102C has a microphone 201 and performs correction of an acoustic characteristic (a frequency characteristic and a phase characteristic) with respect to a sound collected by the chest piece 101 and wirelessly transmits the corrected acoustic signal to the headphone 107M that a user wears. Although detailed explanation is omitted, the acoustic processing unit 102C has the same configuration as the acoustic processing unit 102C in the vibration detecting apparatus 100C illustrated in FIG. 20.

The filtering of the correction filter is performed at the side of the headphone 107M, not the acoustic processing unit 102C. The headphone 107M does not directly perform the filtering of the correction filter and uses a filtering function in an external apparatus on a cloud. In this case, the headphone 107M performs communication for the filtering of the correction filter between the external apparatus on the cloud, that is, the external apparatus connected to a network and the headphone 107M.

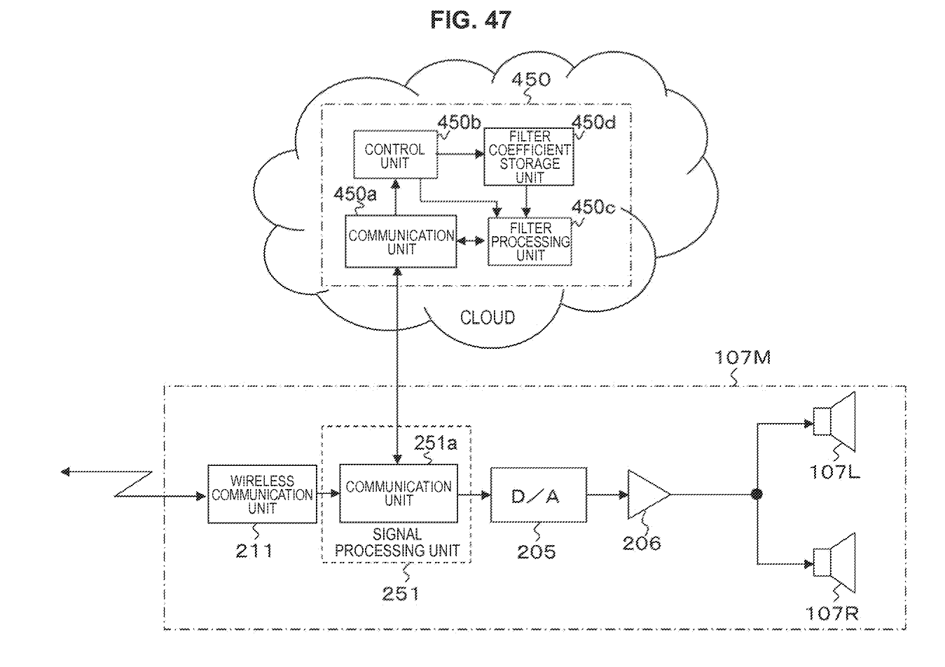

FIG. 47 illustrates a configuration example of the headphone 107M. In FIG. 47, structural elements that correspond to the structural elements of FIGS. 20 and 44 are denoted with the same reference numerals and repeated explanation of these structural elements is omitted. The headphone 107M has a wireless communication unit 211, a signal processing unit 251, a D/A converter 205, an amplifier 206, and speakers 107L and 107R. The wireless communication unit 211 performs communication between the acoustic processing unit 102C and the wireless communication unit 211. That is, the wireless communication unit 211 receives an acoustic signal (vibration signal) that is transmitted from the acoustic processing unit 102C. The wireless communication unit 211 performs communication for wireless connection processing between the acoustic processing unit 102C and the wireless communication unit 211.

The signal processing unit 251 has a communication unit 251a. The communication unit 251a transmits the acoustic signal (vibration signal) received by the wireless communication unit 211 to the external apparatus 450 on the cloud. The communication unit 251a receives the corrected acoustic signal (vibration signal) that is obtained by the external apparatus 450 on the cloud. Although detailed explanation is omitted, the external apparatus 450 has the same configuration as the configuration illustrated in FIG. 44.

The D/A converter 205 converts the corrected acoustic signal (vibration signal) received by the communication unit 251a from a digital signal to an analog signal. The amplifier 206 amplifies the acoustic signal output from the D/A converter 205 and supplies the acoustic signal to the left speaker 107L and the right speaker 107R.

Next, an operation of the headphone 107M illustrated in FIG. 47 will be described. In the wireless communication unit 211 of the headphone 107M, the acoustic signal (vibration signal) that is transmitted from the acoustic processing unit 102C is received and is supplied to the signal processing unit 251. In the signal processing unit 251, the received acoustic signal is transmitted to the external apparatus 450 on the cloud by the communication unit 251a. At this time, the body information or the target information is transmitted together with the acoustic signal.

In the external apparatus 450, filtering of the correction filter based on the filter coefficient (impulse response) selected based on the body information or the target information is performed by the filter processing unit 450c. The acoustic signal is transmitted from the external apparatus 450 to the communication unit 251a. The corrected acoustic signal that is received by the communication unit 251a is converted by the D/A converter 205 from a digital signal to an analog signal, is amplified by the amplifier 206, and is supplied to the speakers 107L and 107R. A sound (vibration) that is obtained from the corrected acoustic signal is output from these speakers 107L and 107R.

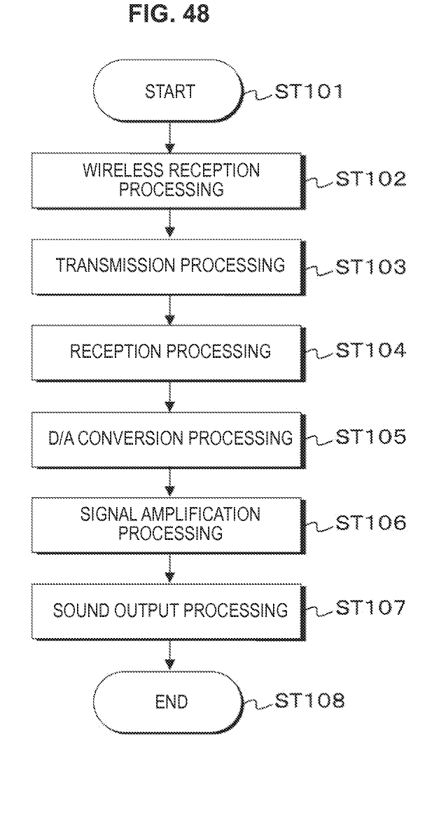

A flowchart of FIG. 48 illustrates a processing sequence of the headphone 107M illustrated in FIG. 47. The headphone 107M starts processing in step ST101 and proceeds to processing of step ST102. In step ST102, the headphone 107M receives the acoustic signal (vibration signal) transmitted from the acoustic processing unit 102C by the wireless communication unit 211. In step ST103, the headphone 107M transmits the received acoustic signal to the external apparatus 450 on the cloud by the communication unit 215a, of the signal processing unit 251.

Next, in step ST104, the headphone 107M receives the corrected acoustic signal transmitted from the external apparatus 450 on the cloud, by the communication unit 215a of the signal processing unit 251. In step ST105, the headphone 107M converts the corrected acoustic signal from a digital signal to an analog signal.

Next, in step ST106 the headphone 107M amplifies the acoustic signal. In step ST107, the headphone 107M outputs a sound (vibration) obtained from the corrected acoustic signal from the speakers 107L and 107R. Then, in step ST108, the headphone 107M ends the processing.

As described above, in the vibration detecting apparatus 100L illustrated in FIG. 46, filtering of a filter characteristic including an inverse characteristic of an entire acoustic characteristic or a partial acoustic characteristic of the chest piece 101, the microphone 201, and the speakers 107L and 107R is executed by the external apparatus 450 on the cloud. For this reason, deterioration of a frequency characteristic in each portion such as the chest piece 101 can be compensated for and a user can hear a biological vibration sound such as a cardiac sound or a pulmonary sound with a superior characteristic, using the headphone 107M.

In the vibration detecting apparatus 100L illustrated in FIG. 46, the headphone 107M does not directly perform the filtering and uses a filtering function in the external apparatus 450 on the cloud. For this reason, a biological vibration signal having a superior characteristic can be obtained without providing a correction filter having a heavy processing load in the headphone 107M.

In the vibration detecting apparatus 100L illustrated in FIG. 46, the acoustic processing unit 102C and the headphone 107M are wirelessly connected. However, instead of the headphone 107M, a display device and the acoustic processing unit 102C may be connected (refer to FIG. 23).

### 14. Fourteenth Embodiment

### Configuration Example of Electronic Medical Chart Generating Apparatus

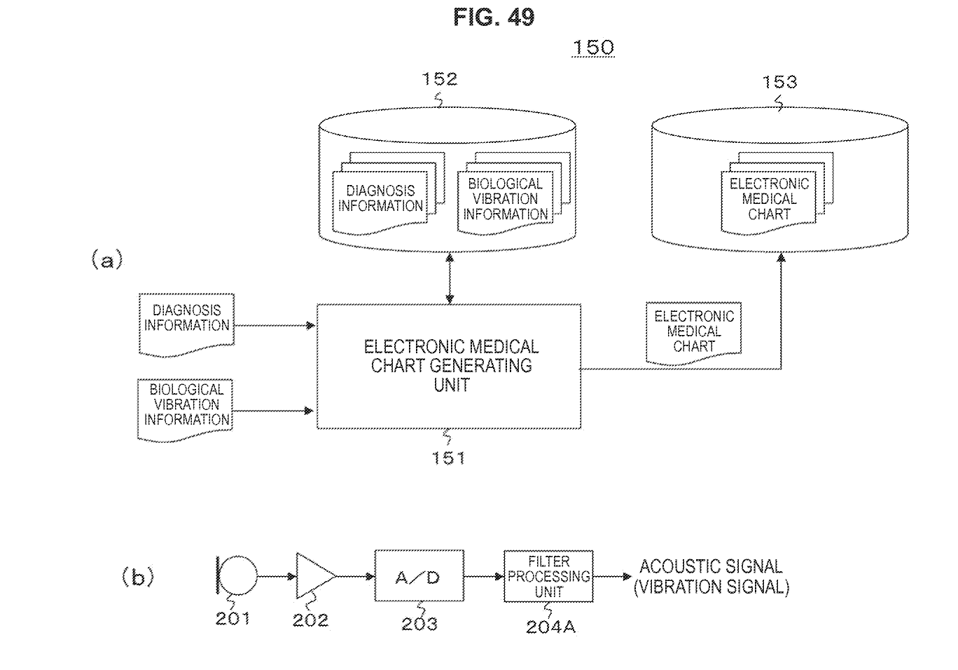

FIG. 49(a) illustrates a configuration example of an electronic medical chart generating apparatus 150 according to a fourteenth embodiment. The electronic medical chart generating apparatus 150 includes an electronic medical chart generating unit 151, a patient information database 152, and an electronic medical chart storage unit 153.

Diagnosis information of a doctor with respect to a patient of an electronic medical chart generation object is input to the electronic medical chart generating unit 151. In addition, biological vibration information of the patient such as a cardiac sound and a pulmonary sound is input to the electronic medical chart generating unit 151. The biological vibration information corresponds to the acoustic signal (vibration signal) that is output from the acoustic processing unit 102B in the vibration detecting apparatus 100 illustrated in FIG. 11 described above.

FIG. 49(b) illustrates a configuration example of the acoustic processing unit 102B (refer to FIG. 12). The acoustic signal that is output from the acoustic processing unit 102B is an acoustic signal that is corrected by performing filtering of an inverse characteristic of an acoustic characteristic of the chest piece 101 and the microphone 201 by the filter processing unit 204A. The acoustic signal is a normalized acoustic signal in which an influence of the acoustic characteristic of the chest piece 101 and the microphone 201 is removed.

In the patient information database 152, diagnosis information and biological vibration information of a plurality of patients are accumulated. The electronic medical chart generating unit 151 collates diagnosis information and biological vibration information of an input object patient with diagnosis information and biological vibration information of other patients accumulated in the patient information database 152 and obtains an automatic diagnosis result of the object patient. The electronic medical chart generating unit 151 generates an electronic medical chart including the input diagnosis information, the input biological vibration information, and the automatic diagnosis result as an electronic medical chart of the object patient and stores the electronic medical chart in the electronic medical chart storage unit 153.

As described above, in the electronic medical chart generating apparatus 150 illustrated in FIG. 49, the biological vibration information corresponds to a normalized acoustic signal (vibration signal). That is, an influence of an acoustic characteristic of a chest piece of a vibration detecting apparatus (digital stethoscope) that is used by each doctor is removed. For this reason, precision of the automatic diagnosis result of the object patient that is obtained by collating the diagnosis information and the biological vibration information of the object patient with the diagnosis information and the biological vibration information of other patients accumulated in the patient information database 152 can be raised.

The series of processes in the electronic medical chart generating apparatus 150 illustrated in FIG. 49 is executed by software. In this case, a program configuring the software is installed in a general-purpose computer.

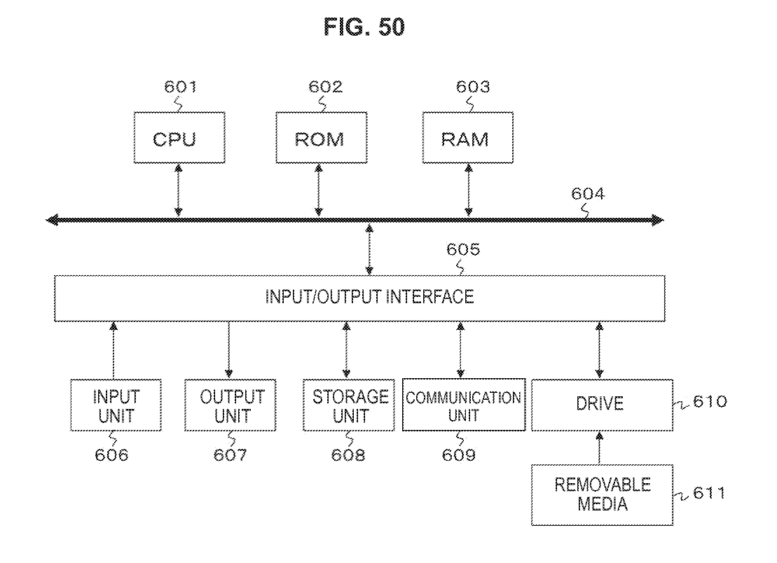

FIG. 50 illustrates a configuration example of the computer in which the program executing the series of processes is installed. The program can be previously recorded in a storage unit 608 or a read on memory (ROM) 602 functioning as recording media embedded in the computer.

The program can be stored (recorded) in removable media 611. The removable media 611 can be provided as so-called package software. In this case, a flexible disc, a compact disc read only memory (CD-ROM), a magneto-optical (MO) disc, a digital versatile disc (DVD), a magnetic disc, and a semiconductor memory are exemplified as the removable media 611.

The program can be installed from the removable media 611 to the computer through a drive 610. In addition, the program can be downloaded to the computer through a communication network or a broadcasting network and can be installed in the embedded storage unit 608. That is, the program can be transmitted by wireless, from a download site to the computer through an artificial satellite for digital satellite broadcasting, or can be transmitted by wire, from the download site to the computer through a network such as a local area network (LAN) or the Internet.

The computer has a central processing unit (CPU) 601 embedded therein and an input/output interface 605 is connected to the CPU 601 through a bus 604. If a command is input to the CPU 601 through the input/output interface 605 by operating an input unit 606 by a user, the CPU 601 executes the program stored in the ROM 602, according to the command. The CPU 601 loads the program stored in the storage unit 608 to a random access memory (RAM) 603 and executes the program.

Thereby, the CPU 601 executes the series of processes executed by the configuration of the block diagram described above. In addition, the CPU 601 outputs the processing result from an output unit 607, transmits the processing result from a communication unit 609, or records the processing result in the storage unit 608, through the input/output interface 605, according to necessity. The input unit 606 is configured using a keyboard, a mouse, and a microphone. The output unit 607 is configured using a liquid crystal display (LCD) and a speaker.

### 15. Fifteenth Embodiment

### Configuration Example of Measurement Supporting Apparatus

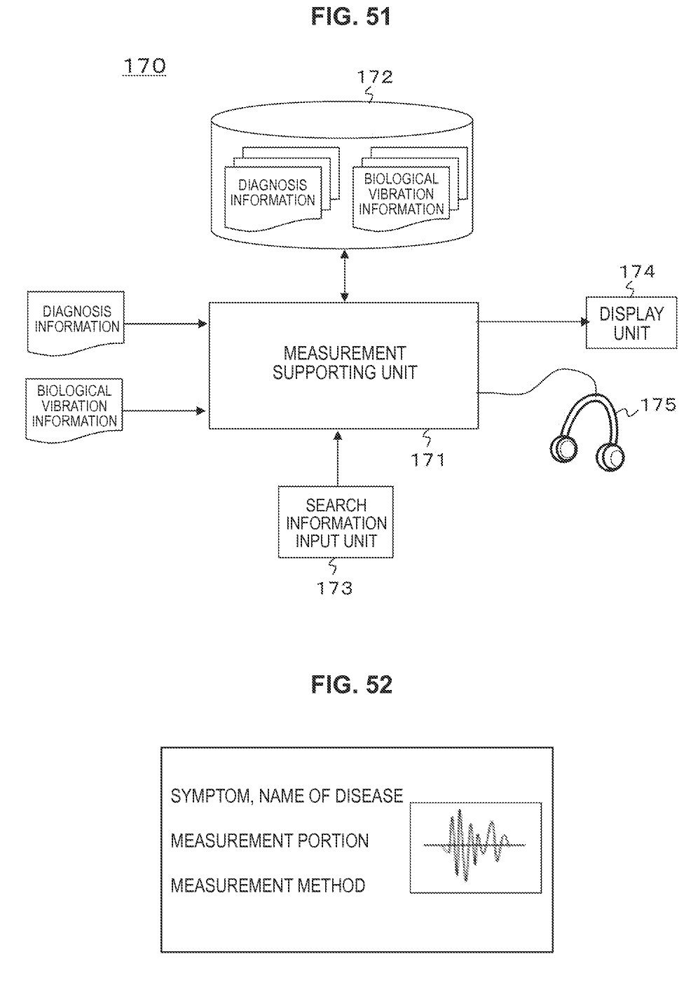

FIG. 51 illustrates a configuration example of a measurement supporting apparatus 170 according to a fifteenth embodiment. The measurement supporting apparatus 170 includes a measurement supporting unit 171, a patient information database 172, a search information input unit 173, a display unit 174, and a headphone 175.

Diagnosis information of a doctor with respect to a patient and biological vibration information of the patient such as a cardiac sound or a pulmonary sound are input to the measurement supporting unit 171. The biological vibration information corresponds to an acoustic signal (vibration signal) that is output from the acoustic processing unit 102B in the vibration detecting apparatus 100 illustrated in FIG. 11, similar to the biological vibration information input to the electronic medical chart generating unit 151 of the electronic medical chart generating apparatus 150 illustrated in FIG. 49. The acoustic signal is a normalized acoustic signal in which an influence of the acoustic characteristic of the chest piece 101 and the microphone 201 is removed.

In the patient information database 172, diagnosis information and biological vibration information of a plurality of patients that are input to the measurement supporting unit 171 described above are accumulated. In this embodiment, not only the acoustic signal (vibration signal) but also information of a measurement portion and a measurement method of the biological vibration is added to the biological vibration information accumulated in the patient information database 172. The added information can be included in the diagnosis information.

The search information input unit 173 is a unit that is used when a user inputs a symptom (for example, “I have a cough”, “I have difficulty in breathing”, and “I have a chest pain”) or a name of disease (for example, “pulmonary tuberculosis”, “bronchitis”, and “cold”). When search information is input from the search information input unit 173, the measurement supporting unit 171 refers to the diagnosis information and the biological vibration information of the plurality of patients accumulated in the patient information database 172 and generates measurement support information corresponding to the input search information.

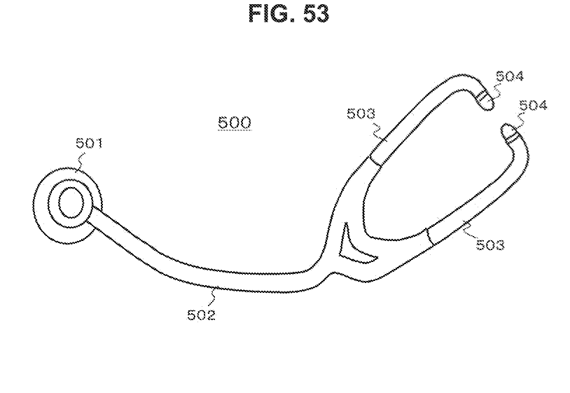

The measurement supporting unit 171 generates a display signal to display the measurement support information and supplies the display signal to the display unit 174. FIG. 52 illustrates an example of the measurement support information that is displayed on the display unit 174. The measurement support information includes information of “a symptom”, “a name of disease”, “a measurement portion”, and a “measurement method” and information of a waveform or a frequency spectrum of an acoustic signal (vibration signal) generally detected by the symptom and the name of diseases. The measurement supporting unit 171 generates the acoustic signal (vibration signal) and supplies the acoustic signal to the headphone 175.

As described above, in the measurement supporting apparatus 170 illustrated in FIG. 51, the acoustic signal (vibration signal) that corresponds to the biological vibration information accumulated in the patient information database 172 is a normalized signal. That is, an influence of an acoustic characteristic of a chest piece of a vibration detecting apparatus (digital stethoscope) that is used by each doctor is removed.

For this reason, the measurement supporting unit 171 can generate appropriate measurement support information by referring to the diagnosis information and the biological vibration information of the plurality of patients accumulated in the patient information database 172. Therefore, a doctor can detect a biological vibration by referring to the measurement support information and obtain an accurate diagnosis result.

The series of processes in the measurement supporting apparatus 170 illustrated in FIG. 51 is executed by software. In this case, a program configuring the software is installed in a general-purpose computer and is executed.

### 16. Modification

In the embodiments described above, the biological vibration detecting unit has the configuration in which the microphone is mounted on the chest piece. However, the configuration of the biological vibration detecting unit is not limited thereto. For example, the biological vibration detecting unit may be configured to use an acceleration sensor that is used in a state in which the acceleration sensor directly adheres closely to a skin and a sensor that detects a vibration from a reflection wave such as a laser or a supersonic wave.

It should be understood by those skilled in the art that various modifications, combinations, sub-combinations and alterations may occur depending on design requirements and other factors insofar as they are within the scope of the appended claims or the equivalents thereof.

Additionally, the present technology may also be configured as below.

(1)
A vibration detecting apparatus including:

a biological vibration detecting unit that is capable of detecting a biological vibration; and

a correction filter that corrects a frequency characteristic and a phase characteristic of a vibration signal obtained by the biological vibration detecting unit with at least an inverse characteristic of a characteristic of the biological vibration detecting unit.

(2)
The vibration detecting apparatus according to (1),

wherein the correction filter is a multi-stage filter that includes a predetermined number of static filters having a fixed filter characteristic and a predetermined number of dynamic filters having a variable filter characteristic.

(3)
The vibration detecting apparatus according to (1) or (2),

wherein the correction filter is a filter that has a constant group delay characteristic.

(4)
The vibration detecting apparatus according to any one of (1) to (3), further including:

a filter characteristic switching unit that switches a filter characteristic of the correction filter.

(5)
The vibration detecting apparatus according to (4),

wherein the filter characteristic switching unit switches the filter characteristic using a filter coefficient downloaded from an external apparatus connected to a network.

(6)
The vibration detecting apparatus according to any one of (1) to (5), further including:

a sound output unit that outputs a sound corresponding to the vibration signal corrected by the correction filter.

(7)

The vibration detecting apparatus according to (6),

wherein the biological vibration detecting unit has a plurality of independent detecting units,

wherein the correction filter corrects vibration signals obtained by the plurality of detecting units with respective filter characteristics, and

wherein the sound output unit selectively outputs at least sounds corresponding to the plurality of vibration signals corrected by the correction filter.

(8)
The vibration detecting apparatus according to any one of (1) to (7), further including:

a display unit that displays a waveform and/or a frequency spectrum corresponding to the vibration signal corrected by the correction filter.

(9)
The vibration detecting apparatus according to any one of (1) to (8), further including:

a wireless transmitting unit that wirelessly transmits the vibration signal corrected by the correction filter to a predetermined number of external apparatuses.

(10)
The vibration detecting apparatus according to (9),

wherein the wireless transmitting unit selectively performs wireless transmission with respect to a second external apparatus, based on an operation signal in a first external apparatus.

(11)
The vibration detecting apparatus according to any one of (1) to (10),

wherein the biological vibration detecting unit has a configuration in which a microphone is mounted on a chest piece.

(12)
A vibration detecting method including:

detecting a biological vibration by a biological vibration detecting unit and obtaining a vibration signal; and

correcting a frequency characteristic and a phase characteristic of the vibration signal with at least an inverse characteristic of a characteristic of the biological vibration detecting unit.

(13)
A program for causing a computer to function as:

a correction filter mechanism for correcting a frequency characteristic and a phase characteristic of a vibration signal obtained by detection of a biological vibration detecting unit with at least an inverse characteristic of a characteristic of the biological vibration detecting unit.

(14)
A vibration detecting apparatus including

a wireless receiving unit that receives a vibration signal obtained by detection of a biological vibration detecting unit; and

a correction filter that corrects a frequency characteristic and a phase characteristic of the received vibration signal with at least an inverse characteristic of a characteristic of the biological vibration detecting unit.

(15)
The vibration detecting apparatus according to (14), further including:

a filter characteristic switching unit that switches a filter characteristic of the correction filter.

(16)
The vibration detecting apparatus according to (15),

wherein the filter characteristic switching unit switches the filter characteristic using a filter coefficient downloaded from an external apparatus connected to a network.

(17)
The vibration detecting apparatus according to (15) or (16),

wherein, when the wireless receiving unit is wirelessly connected to a wireless transmitting apparatus transmitting the vibration signal, the filter characteristic switching unit acquires filter characteristic information of the correction filter from the wireless transmitting apparatus and switches the filter characteristic of the correction filter.

(18)
A vibration detecting apparatus including

a vibration signal acquiring unit that acquires a vibration signal obtained by detection of a biological vibration detecting unit; and

a signal processing unit that outputs a result that is obtained by performing filtering of a correction filter correcting a frequency characteristic and a phase characteristic with at least an inverse characteristic of a characteristic of the biological vibration detecting unit with respect to the vibration signal,

wherein the signal processing unit includes a communication unit that performs communication for the filtering with an external apparatus connected to a network.

(19)
A vibration detecting system including:

a transmission-side apparatus; and

a reception-side apparatus,

wherein the transmission-side apparatus includes a biological vibration detecting unit that is capable of detecting a biological vibration, a correction filter that corrects a frequency characteristic and a phase characteristic of a vibration signal with at least an inverse characteristic of a characteristic of the biological vibration detecting unit, and a wireless transmitting unit that wirelessly transmits the corrected vibration signal to the reception-side apparatus, and

wherein the reception-side apparatus includes a wireless receiving unit that receives the wirelessly transmitted vibration signal and a vibration signal using unit that uses the received vibration signal.

(20)
A vibration detecting system including:

a transmission-side apparatus; and

a reception-side apparatus,

wherein the transmission-side apparatus includes a biological vibration detecting unit that is capable of detecting a biological vibration and a wireless transmitting unit that wirelessly transmits a vibration signal obtained by the biological vibration detecting unit to the reception-side apparatus, and

wherein the reception-side apparatus includes a wireless receiving unit that

receives the wirelessly transmitted vibration signal, a correction filter that corrects a frequency characteristic and a phase characteristic of the received vibration signal with at least an inverse characteristic of a characteristic of the biological vibration detecting unit, and a vibration signal using unit that uses the corrected vibration signal.

The present disclosure contains subject matter related to that disclosed in Japanese Priority Patent Application JP 201.2-178558 filed in the Japan Patent Office on Aug. 10, 2012, the entire content of which is hereby incorporated by reference.

What is claimed is:

1. A vibration detecting apparatus comprising:
a biological vibration detecting unit that is capable of detecting a biological vibration; and
a correction filter that corrects a frequency characteristic and a phase characteristic of a vibration signal obtained by the biological vibration detecting unit with at least an inverse characteristic of a characteristic of the biological vibration detecting unit.

2. The vibration detecting apparatus according to claim 1,
wherein the correction filter is a multi-stage filter that includes a predetermined number of static filters having a fixed filter characteristic and a predetermined number of dynamic filters having a variable filter characteristic.

3. The vibration detecting apparatus according to claim 1,
wherein the correction filter is a filter that has a constant group delay characteristic.

4. The vibration detecting apparatus according to claim 1, further comprising:
a filter characteristic switching unit that switches a filter characteristic of the correction filter.

5. The vibration detecting apparatus according to claim 4,
wherein the filter characteristic switching unit switches the filter characteristic using a filter coefficient downloaded from an external apparatus connected to a network.

6. The vibration detecting apparatus according to claim 1, further comprising:
a sound output unit that outputs a sound corresponding to the vibration signal corrected by the correction filter.

7. The vibration detecting apparatus according to claim 6,
wherein the biological vibration detecting unit has a plurality of independent detecting units,
wherein the correction filter corrects vibration signals obtained by the plurality of detecting units with respective filter characteristics, and
wherein the sound output unit selectively outputs at least sounds corresponding to the plurality of vibration signals corrected by the correction filter.

8. The vibration detecting apparatus according to claim 1, further comprising:
a display unit that displays a waveform and/or a frequency spectrum corresponding to the vibration signal corrected by the correction filter.

9. The vibration detecting apparatus according to claim 1, further comprising:
a wireless transmitting unit that wirelessly transmits the vibration signal corrected by the correction filter to a predetermined number of external apparatuses.

10. The vibration detecting apparatus according to claim 9,
wherein the wireless transmitting unit selectively performs wireless transmission with respect to a second external apparatus, based on an operation signal in a first external apparatus.

11. The vibration detecting apparatus according to claim 1,
wherein the biological vibration detecting unit has a configuration in which a microphone is mounted on a chest piece.

12. A vibration detecting method comprising:
detecting a biological vibration by a biological vibration detecting unit and obtaining a vibration signal; and
correcting a frequency characteristic and a phase characteristic of the vibration signal with at least an inverse characteristic of a characteristic of the biological vibration detecting unit.

13. A program for causing a computer to function as:
a correction filter mechanism for correcting a frequency characteristic and a phase characteristic of a vibration signal obtained by detection of a biological vibration detecting unit with at least an inverse characteristic of a characteristic of the biological vibration detecting unit.

14. A vibration detecting apparatus comprising:
a wireless receiving unit that receives a vibration signal obtained by detection of a biological vibration detecting unit; and
a correction filter that corrects a frequency characteristic and a phase characteristic of the received vibration signal with at least an inverse characteristic of a characteristic of the biological vibration detecting unit.

15. The vibration detecting apparatus according to claim 14, further comprising:
a filter characteristic switching unit that switches a filter characteristic of the correction filter.

16. The vibration detecting apparatus according to claim 15,
wherein the filter characteristic switching unit switches the filter characteristic using a filter coefficient downloaded from an external apparatus connected to a network.

17. The vibration detecting apparatus according to claim 15,
wherein, when the wireless receiving unit is wirelessly connected to a wireless transmitting apparatus transmitting the vibration signal, the filter characteristic switching unit acquires filter characteristic information of the correction filter from the wireless transmitting apparatus and switches the filter characteristic of the correction filter.

18. A vibration detecting apparatus comprising:
a vibration signal acquiring unit that acquires a vibration signal obtained by detection of a biological vibration detecting unit; and
a signal processing unit that outputs a result that is obtained by performing filtering of a correction filter correcting a frequency characteristic and a phase characteristic with at least an inverse characteristic of a characteristic of the biological vibration detecting unit with respect to the vibration signal,
wherein the signal processing unit includes a communication unit that performs communication for the filtering with an external apparatus connected to a network.

19. A vibration detecting system comprising:
a transmission-side apparatus; and
a reception-side apparatus,
wherein the transmission-side apparatus includes a biological vibration detecting unit that is capable of detecting a biological vibration, a correction filter that corrects a frequency characteristic and a phase characteristic of a vibration signal with at least an inverse characteristic of a characteristic of the biological vibration detecting unit, and a wireless transmitting unit that wirelessly transmits the corrected vibration signal to the reception-side apparatus, and
wherein the reception-side apparatus includes a wireless receiving unit that receives the wirelessly transmitted vibration signal and a vibration signal using unit that uses the received vibration signal.

20. A vibration detecting system comprising:
a transmission-side apparatus; and
a reception-side apparatus,
wherein the transmission-side apparatus includes a biological vibration detecting unit that is capable of detecting a biological vibration and a wireless transmitting unit that wirelessly transmits a vibration signal obtained by the biological vibration detecting unit to the reception-side apparatus, and
wherein the reception-side apparatus includes a wireless receiving unit that receives the wirelessly transmitted vibration signal, a correction filter that corrects a frequency characteristic and a phase characteristic of the received vibration signal with at least an inverse characteristic of a characteristic of the biological vibration detecting unit, and a vibration signal using unit that uses the corrected vibration signal.
